SEQUENCE LISTING (72 sequences)

SEQ ID NO: 1 - protein, 111 aa, Mus musculus
RHHLPDRSQLCRRVKFQVDFNLIGWGSWIIYPKQYNAYRCEGECPNPVGEEFHPTNHAYIQSLLKRYQPHRVPSTCCAPVKTKPLSMLYVDNGRVLLEHHKDMIVEECGCL

SEQ ID NO: 2 - protein, 21 aa, Mus musculus
RHHLPDRSQLCRRVKFQVDFN

SEQ ID NO: 3 - protein, 7 aa, Mus musculus
YRCEGEC

SEQ ID NO: 4 - protein, 17 aa, Mus musculus
YQPHRVPSTCCAPVKTK

SEQ ID NO: 5 - protein, 11 aa, Mus musculus
KDMIVEECGCL

SEQ ID NO: 6 - protein, 16 aa, Mus musculus
LIGWGSWIIYPKQYNA

SEQ ID NO: 7 - protein, 22 aa, Mus musculus
PNPVGEEFHPTNHAYIQSLLKR

SEQ ID NO: 8 - protein, 17 aa, Mus musculus
PLSMLYVDNGRVLLEHH

SEQ ID NO: 9 - protein, 111 aa, Homo sapiens
RHHLPDRSQLCRKVKFQVDFNLIGWGSWIIYPKQYNAYRCEGECPNPVGEEFHPTNHAYIQSLLKRYQPHRVPSTCCAPVKTKPLSMLYVDNGRVLLDHHKDMIVEECGCL

SEQ ID NO: 10 - protein, 112 aa, Artificial Sequence
  chemically synthesized
RHHLPDRSQLCRRVKFQVDFNDVGWNDWIVAPPGYHYRCEGECPFPLADHLNSTNHAIVQTLVNSYQPHRVPSTCCAPVKTKSAISMLYLDENEKVVLKNYKDMIVEECGCL

SEQ ID NO: 11 - protein, 106 aa, Artificial Sequence
  chemically synthesized
RHHLPDRSQLCRRVKFQVDFNAFGWDWIIAPKRYKYRCEGECPEFVFLQKYPHTHLVQHYQPHRVPSTCCAPVKTKSPINMLYFNGKQQIIYGKIKDMIVEECGCL

SEQ ID NO: 12 - protein, 113 aa, Artificial Sequence
  chemically synthesized
RHHLPDRSQLCRRVKFQVDFNDIGWNDWIIAPSGYHYRCEGECPSHIAGTSGSSLSFHSTVINHYRYQPHRVPSTCCAPVKTKRPMSMLYYDDGQNIIKKDIKDMIVEECGCL

SEQ ID NO: 13 - protein, 112 aa, Artificial Sequence
  chemically synthesized
RHHLPDRSQLCRRVKFQVDFNDLGWHKWIIAPCGFMYRCEGECPFSLTISLNSSNYAFMQALMHAYQPHRVPSTCCAPVKTKSPISMLYQDNNDNVILRHYKDMIVEECGCL

SEQ ID NO: 14 - protein, 111 aa, Artificial Sequence
  chemically synthesized
RHHLPDRSQLCRRVKFQVDFNEIGWDSWIIAPPGYEYRCEGECYPLAEHLTPTKHAIIQALVHLYQPHRVPSTCCAPVKTKEPISILYLDKGVVTYKFKYKDMIVEECGCL

SEQ ID NO: 15 - protein, 103 aa, Artificial Sequence
  chemically synthesized
RHHLPDRSQLCRRVKFQVDFNAFGWDWIIAPKRYKYRCEGECEFVFLQKYPHTHLVHQYQPHRVPSTCCAPVKTKSPINMLYFNGKEQIIYGKIKDMIVEECG

SEQ ID NO: 16 - protein, 111 aa, Artificial Sequence
  chemically synthesized
RHHLPDRSQLCRRVKFQVDFNDMGWDDWIIAXLEYEYRCEGECEFPLRSHLEPTNHAVIQTLMNSYQPHRVPSTCCAPVKTKSPISILFIDSANNVVKQYKDMIVEECGCL

SEQ ID NO: 17 - protein, 112 aa, Artificial Sequence
  chemically synthesized
RHHLPDRSQLCRRVKFQVDFNELGWDDWIIAPLEYEYRCEGECDFPLRSHLEPTNHAIIQTLMNSYQPHRVPSTCCAPVKTKTPISILYIDAGNNVVYNEYKDMIVEECGCL

SEQ ID NO: 18 - protein, 112 aa, Artificial Sequence
  chemically synthesized
RHHLPDRSQLCRRVKFQVDFNELGWDDWIIAPLDYEYRCEGECDFPLRSHLEPTNHAIIQTLLNSYQPHRVPSTCCAPVKTKSPISILYIDAANNVVYKQYKDMIVEECGCL

SEQ ID NO: 19 - protein, 111 aa, Artificial Sequence
  chemically synthesized
RHHLPDRSQLCRRVKFQVDFNEIGWDSWIIAPPGYEYRCEGECNYPLAEHLTPTKHAIIQALVHLYQPHRVPSTCCAPVKTKEPISILYLDKGVVTYKFYKDMIVEECGCL

SEQ ID NO: 20 - protein, 112 aa, Artificial Sequence
  chemically synthesized
RHHLPDRSQLCRRVKFQVDFNDVGWNDWIVAPPGYQYRCEGECPFPLADHLNSTNHAIVQTLVNSYQPHRVPSTCCAPVKTKSAISMLYLDEYDKVVLKNYKDMIVEECGCL

SEQ ID NO: 21 - protein, 109 aa, Artificial Sequence
  chemically synthesized
RHHLPDRSQLCRRVKFQVDFNDLGWQDWIIAPEGYAYRCEGECAFPLNSYMNATNHAIVQTLVHFYQPHRVPSTCCAPVKTKNAISVLYFDDSSNVILKDMIVEECGCL

SEQ ID NO: 22 - protein, 112 aa, Artificial Sequence
  chemically synthesized
RHHLPDRSQLCRRVKFQVDFNDLGWQDWIIAPKGYAYRCEGECSFPLNAHMNATNHAIVQTLVHLYQPHRVPSTCCAPVKTKNAISVLYFDDNSNVILKKYKDMIVEECGCL

SEQ ID NO: 23 - protein, 109 aa, Artificial Sequence
  chemically synthesized
RHHLPDRSQLCRRVKFQVDFNDLGWQDWIIAPEGYAYRCEGECSFPNLAHMNATNHAIVQTLVHLYQPHRVPSTCCAPVKTKNAISVLYFDDSSNVILKDMIVEECGCL

SEQ ID NO: 24 - protein, 112 aa, Artificial Sequence
  chemically synthesized
RHHLPDRSQLCRRVKFQVDFNDLGWLDWVIAPQGYSYRCEGECSFPLDSCMNATNHAILQSLVHLYQPHRVPSTCCAPVKTKSATSVLYYDSSNNVILRKHKDMIVEECGCL

SEQ ID NO: 25 - protein, 117 aa, Artificial Sequence
  chemically synthesized
RHHLPDRSQLCRRVKFQVDFNEVGWHRWVIAPRGFLYRCEGECALPVALSGSGGPPALNHAVLRALMHAAYQPHRVPSTCCAPVKTKSPISVLFFDNSDNVVLRQYKDMIVEECGCL

SEQ ID NO: 26 - protein, 112 aa, Artificial Sequence
  chemically synthesized
RHHLPDRSQLCRRVKFQVDFNDIGWSEWIISPKSFDYRCEGECQFPMPKFLKPSNHATIQSIVRAYQPHRVPSTCCAPVKTKSSLSILFFDENKNVVLKVYKDMIVEECGCL

SEQ ID NO: 27 - protein, 107 aa, Artificial Sequence
  chemically synthesized
RHHLPDRSQLCRRVKFQVDFNDLGWKWIHTKGYHYRCEGECPYIWSLDTQYSKVLALYNQHYQPHRVPSTCCAPVKTKEPLPIVYYVGRKPKVEQLKDMIVEECGCL

SEQ ID NO: 28 - protein, 113 aa, Artificial Sequence
  chemically synthesized
RHHLPDRSQLCRRVKFQVDFNQLGWDHWIIAPPFYTYRCEGECLRVLRDGLNSPNHAIIQNLINQLYQPHRVPSTCCAPVKTKVPISVLMIEANGSILYKEYKDMIVEECGCL

SEQ ID NO: 29 - protein, 113 aa, Artificial Sequence
  chemically synthesized
RHHLPDRSQLCRRVKFQVDFNQLKWDNWIVAPHRYNYRCEGECPRAVGHRYGSPVHTMVQNIIYEKYQPHRVPSTCCAPVKTKSPLSVLTIEPDGSIAYKEYKDMIVEECGCL

SEQ ID NO: 30 - protein, 109 aa, Artificial Sequence
  chemically synthesized
RHHLPDRSQLCRRVKFQVDFNDLGWADWVLSPREVQYRCEGECPSQFRAANMHAQIKTSLHRLYQPHRVPSTCCAPVKTKNPMVLIQKTDTGVSLQTYKDMIVEECGCL

SEQ ID NO: 31 - protein, 113 aa, Artificial Sequence
  chemically synthesized
RHHLPDRSQLCRRVKFQVDFNGMKAENWVLEPPGFLYRCEGECPRAVGHRYGSPVHTMVQNIIYEKYQPHRVPSTCCAPVKTKSPLSVLTIEPDGSIAYKEYKDMIVEECGCL

SEQ ID NO: 32 - DNA, 64 bp, Mus musculus
cgacatcatttgccagacagaagccaactgtgtaggagggtcaagttccaggtggacttcaacc

SEQ ID NO: 33 - DNA, 21 bp, Mus musculus
tatcgctgtgagggcgagtgt

SEQ ID NO: 34 - DNA, 51 bp, Mus musculus
taccaaccccaccgggttccttccacgtgctgtgcccccgtgaagaccaag

SEQ ID NO: 35 - DNA, 33 bp, Mus musculus
aaggacatgattgtggaggagtgtgggtgcctc

SEQ ID NO: 36 - DNA, 2160 bp, Mus musculus
aagcttcactcggagcaagccttagcccgctgtctcagcagggagacttcccgaggtagaggggcaaggtgcggggcggtttagactcagagtctgtatgcacccctaactcccccccccccccccgccacaatttctctgtagtctttctcagcacatcacacctcccctcagcaggggctcccctgccctgcccctccagggtggttataagttcttaacctataggttataggcctctccggagggagggagggaaaggggcggggcggcggcggctcagatataagggaccctggtgttgtttgcgggtccaaacagcccaccatgagtgcccacagcctccgcatccttcttcttcaagcctgttgggctctactccacccgcgcgccccgaccgcggccgctttgcctctgtggacacgggggcagccctcgtcaccgtcccctctggcgtacatgttgagcctctaccgagacccgctgcctcgggcggacatcatccgcagcctccaggcgcaagatgtggacgtgaccggacagaactggactttcacgtttgacttctcctttttgagccaagaagaggatctggtatgggcggacgtccggttgcagctgccgggccccatggacatacccactgagggcccactcaccattgacattttccaccaggccaagggggatccagagcgggaccccgctgactgcctggagcgcatttggatggagacgttcaccgtcattccttctcaggtcacgtttgcctcaggcagcacagtcctggaggtgaccaagccactctccaagtggctaaaggaccccagggcactggaaaagcaggtgtccagtcgagcagaaaagtgttggcatcagccctacaccccacctgtacctgtcgccagcaccaatgtgctcatgctctactccaaccggcctcaggagcagaggcagctagggggcgccactttgctttgggaagctgagagctcctggcgggcccaggagggacagctgtctgtagagaggggcggatggggcagaaggcaacgccgacatcatttgccagacagaagccaactgtgtaggagggtcaagttccaggtggacttcaacctgattggctggggctcctggatcatctaccccaagcagtacaatgcctatcgctgtgagggcgagtgtcctaaccctgtgggggaggagtttcatcctaccaaccatgcctacatccagagcctgctgaaacgataccaaccccaccgggttccttccacgtgctgtgcccccgtgaagaccaagccactgagcatgctttatgtggacaatggcagggtcctcctggaacaccacaaggacatgattgtggaggagtgtgggtgcctctgacagagccagggggagtgctgaaattggcttgcattccacaatgctgatgaactccaaggagactccattgtgtctatccagggagcagaaacgttagaagagttctgcctgctggagctaaagagaaaagccccgccccctgtgcatacagtgctcttagacctgccaagccagagagaggctaccgtggcatggcaggatggggaagccttgcaggggctggctcgctgggctccctggaaatagggtttatgaactgcttgaaattgtgtgcaaaggctggggtgtatatatatatatgtatatatatatatatgtatttatttttgatgccatctgtaactttcttgtattctcaaaagtggtctgtgacctgctgtccctccctcaagattagtatatattttattagattataaacgagccatttggttctccctgcctcaagctgtggtagggaagacccacaaccttctggctggctggcagtgacatcctggccttggtcaggggctctctgatctctaatgacttgcctaaaaaagccactgtccagttctccagggccagttggtgcctttgaccagagaggtgggcacttgtccaagaggggactggccatggtggactttagaagccagagtcctgagatgtatgcttggcagacacaacccaagtctattaaaagtctgtgacaattcaaaaaaaaaaaaaaaaaaaaaaaaaaaaaaaaaaaaaaaaaaaaaaaactaaaaaaaaaaaaaaaaa

SEQ ID NO: 37 - protein, 110 aa, Homo sapiens
RNLGLDCDEHSTESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 38 - protein, 110 aa, Homo sapiens
RDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 39 - protein, 110 aa, Homo sapiens
RPSKNKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 40 - protein, 111 aa, Homo sapiens
RSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 41 - protein, 109 aa, Homo sapiens
RNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 42 - protein, 115 aa, Homo sapiens
RAAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 43 - protein, 117 aa, Homo sapiens
REKQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 44 - protein, 105 aa, Homo sapiens
KNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 45 - protein, 118 aa, Homo sapiens
RMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 46 - protein, 118 aa, Homo sapiens
RVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 47 - protein, 118 aa, Homo sapiens
RMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 48 - protein, 119 aa, Homo sapiens
RLPGIFDDVHDSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 49 - protein, 120 aa, Homo sapiens
RDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 50 - protein, 112 aa, Homo sapiens
RKQWDEPRVCSRRYLKVDFADIGWNEWIISPKSFDAYYCAGACEFPMPKIVRPSNHATIQSIVRAVGIIPGIPEPCCVPDKMNSLGVLFLDENRNVVLKVYPNMSVDTCACR

SEQ ID NO: 51 - protein, 111 aa, Homo sapiens
KQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 52 - protein, 111 aa, Homo sapiens; identical to SEQ ID NO: 9

SEQ ID NO: 53 - protein, 117 aa, Homo sapiens
RGIDCQGGSRMCCRQEFFVDFREIGWHDWIIQPEGYAMNFCIGQCPLHIAGMPGIAASFHTAVLNLLKANTAAGTTGGGSCCVPTARRPLSLLYYDRDSNIVKTDIPDMVVEACGCS

SEQ ID NO: 54 - protein, 115 aa, Homo sapiens
RTPTCEPATPLCCRRDHYVDFQELGWRDWILQPEGYQLNYCSGQCPPHLAGSPGIAASFHSAVFSLLKANNPWPASTSCCVPTARRPLSLLYLDHNGNVVKTDVPDMVVEACGCS

SEQ ID NO: 55 - protein, 116 aa, Homo sapiens
RGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 56 - protein, 127 aa, Homo sapiens
RQADGISAEVTASSSKHSVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 57 - protein, 113 aa, Homo sapiens
RALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 58 - protein, 113 aa, Homo sapiens
RALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 59 - protein, 113 aa, Homo sapiens
RALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 60 - protein, 122 aa, Homo sapiens
GISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 61 - protein, 122 aa, Homo sapiens
GPASFNLSEYFRQFLLPQNECELHDFRLSFSQLKWDNWIVAPHRYNPRYCKGDCPRAVGHRYGSPVHTMVQNIIYEKLDSSVPRPSCVPAKYSPLSVLTIEPDGSIAYKEYEDMIATKCTCR

SEQ ID NO: 62 - protein, 95 aa, Homo sapiens
GTRCCRQEMYIDLQGMKWAENWVLEPPGFLAYECVGTCRQPPEALAFKWPFLGPRQCIASETDSLPMIVSIKEGGRTRPQVVSLPNMRVQKCSCA

SEQ ID NO: 63 - protein, 102 aa, Homo sapiens
DGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 64 - protein, 115 aa, Homo sapiens
RARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 65 - protein, 101 aa, Homo sapiens
GRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 66 - protein, 93 aa, Homo sapiens
SGPCQLWSLTLSVAELGLGYASEEKVIFRYCAGSCPRGARTQHGLALARLQGQGRAHGGPCCRPTRYTDVAFLDDRHRWQRLPQLSAAACGCG

SEQ ID NO: 67 - protein, 98 aa, Homo sapiens
ARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 68 - protein, 96 aa, Homo sapiens
NRGCVLTAIHLNVTDLGLGYETKEELIFRYCSGSCDAAETTYDKILKNLSRNRRLVSDKVGQACCRPAFDDDLSFLDDNLVYHILRKHSAKRCGCI

SEQ ID NO: 69 - protein, 15 aa, Homo sapiens
DVGWNDWIVAPPGYH

SEQ ID NO: 70 - protein, 16 aa, Homo sapiens
SDVGWNDWIVAPPGYH

SEQ ID NO: 71 - protein, 14 aa, Homo sapiens
VGWNDWIVAPPGYH

SEQ ID NO: 72 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

(19) United States
(12) Patent Application Publication
(10) Pub. No.: US 2009/0042780 A1
(43) Pub. Date: Feb. 12, 2009

(54) Modified TGF-Beta Superfamily Polypeptides and Related Methods

(75) Inventors: John Knopf, Carlisle, MA (US); Jasbir Seehra, Lexington, MA (US)

Correspondence Address: ROPES & GRAY LLP, PATENT DOCKETING 39/41, ONE INTERNATIONAL PLACE, BOSTON, MA, 02110-2624, US

(73) Assignee: ACCELERON PHARMA INC, Cambridge, MA (US)

(21) Appl. No.: 11/596,801

(22) PCT Filed: May 20, 2005
(86) PCT No.: PCT/US05/17805

§ 371 Date: Jul. 10, 2008

Related U.S. Application Data
(60) Provisional application No. 60/573,718, filed on May 20, 2004.

(51) Int. Cl. A61K 38/18; C07K 14/495; C07H 21/04; C07K 16/00; A61P 9/00; C12N 5/10; C12P 21/00

(52) U.S. Cl. 514/12; 530/402; 536/23.5; 435/325; 435/69.1; 530/391.1

(57) ABSTRACT

The disclosure relates to modified TGF-beta polypeptides. Certain modified polypeptides disclosed herein are chimeric, containing a conserved framework portion from one protein of this family (e.g., Nodal) and variable loops that mediate receptor interaction derived from a second protein of this family (e.g., BMP-2). The chimeras will generally be designed so as to mimic a biological effect of the family member from which the variable loops are derived. Other modified TGF-polypeptides disclosed herein have one or more post-translational modifications that may be situated in one or more core domains.

### BACKGROUND OF THE DISCLOSURE

The transforming growth factor-beta (TGF-beta or TGF-β) superfamily contains many member proteins that share common sequence elements and structural motifs. These proteins are known to elicit a wide spectrum of biological responses in a variety of cell types. Cellular signaling triggered by members of the TGF-beta superfamily members involves cooperative binding of the ligand to both type 1 and type 2 transmembrane receptor components, which induces assembly of an active serine/threonine kinase receptor complex. This receptor complex initiates a signal transduction pathway by phosphorylating cytoplasmic Smad proteins, which then translocate to the nucleus and act to suppress or activate transcription of target genes.

Many TGF-beta superfamily proteins have important functions during embryonic development in pattern formation and tissue specification. TGF-beta superfamily protein-induced signaling regulates a variety of differentiation processes, including adipogenesis, myogenesis, chondrogenesis, hematopoiesis, and epithelial cell differentiation (for review, see Massague, Cell 49:437, 1987; Siegel et al., Nature Review Cancer, October 2003, 8:807-20). In adult tissues, TGF-beta superfamily proteins are also involved in processes such as wound healing, bone repair, and bone remodeling.

The superfamily can be divided into two general branches: the BMP/GDF and the TGF-β/Activin/Nodal branches, member proteins of which have diverse, often complementary, but sometimes opposite effects. Thus, it is desirable to make novel proteins that may be ligands for TGF-beta receptors and/or can mimic, potentiate, or inhibit a particular TGF-beta superfamily member.

### BRIEF DESCRIPTION OF THE DISCLOSURE

In certain aspects, the disclosure provides TGF-beta superfamily proteins that are modified with respect to certain domains referred to herein as the “core” and “variable” domains. Modified TGF-beta superfamily proteins disclosed herein include chimeric forms comprising one or more core domains from a first TGF-beta family member and one or more variable domains from a heterologous source, such as a second TGF-beta superfamily member. Chimeric TGF-beta proteins described herein will generally act as agonists for the signaling pathway that is normally activated by a TGF-beta superfamily member from which one or more of the variable domains are derived. Modified TGF-beta superfamily proteins disclosed herein also include forms having one or more post-translational modifications in one or more of the core domains. Such modifications may be designed to provide advantageous pharmacokinetic properties while preferably having no deleterious effect on the activity of the modified TGF beta polypeptide. Further provided are nucleic acids encoding the modified TGF-beta proteins as well as methods of making and using the modified proteins.

In certain aspects, the disclosure provides “core” domains and “variable” domains of mature polypeptides from the TGF-beta superfamily. Core domains provide a structural framework while variable domains provide various biological functions, including receptor binding and binding to certain inhibitors. Accordingly, it is possible to create a TGF-beta superfamily having a post-translational modification in a core domain without eliminating the biological functionality of the protein. It is also possible, according to the teachings herein, to generate a chimera comprising core domains from one TGF-beta superfamily member and variable domains from a second TGF-beta superfamily member. Such a chimera is expected to have one or more biological activities (e.g., receptor binding) that are similar to those of the second TGF-beta superfamily member, while the core domains provide the structural framework.

In certain embodiments, the disclosure features a TGF-beta superfamily member protein having a modification in a core domain. The TGF-beta superfamily member may comprise a naturally-occurring amino acid sequence or a variant amino acid sequence. The modification may comprise glycosylation of an amino acid of the core domain. Alternatively, the modification may comprise any post-translational modification, such as for example, phosphorylation, PEGylation, farnesylation, acetylation, biotinylation, lipidation (amino acid conjugated with lipid), and/or conjugation with an organic derivatizing agent. Preferably, the modified protein retains one or more biological activities of the unmodified proteins. For example, a modified protein may retain dimerization and receptor activation properties similar to the unmodified protein. This type of modified protein may act as a mimic, or agonist, of the unmodified protein A modified protein may be defective in binding to receptors but may retain the ability to bind to one or more inhibitors. This type of modified protein may act as an agonist of the unmodified protein by competing for binding to the one or more inhibitors. A modified protein may bind to one or more receptors but fail to trigger an appropriate level of signal transduction; this type of modified protein may act as antagonist of the unmodified protein. Biological activities, such as receptor binding, dimerization, inhibitor binding and signal transduction activation are readily assessed by a variety of techniques that are known in the art.

In certain embodiments, the disclosure features a chimeric TGF-beta superfamily protein comprising a core domain from a first TGF-beta superfamily member and a variable domain from a second TGF-beta superfamily member. The chimeric protein may be an agonist of the second TGF-beta superfamily member. Agonist forms will generally retain dimerization and receptor binding activities that are similar to those of the second TGF-beta superfamily member. Alternatively, the chimeric protein may be an antagonist of the second TGF-beta superfamily member.

Antagonists may, for example, compete for binding to one or more receptors but fail to form a complex with the components or conformation necessary for triggering a signal transduction cascade. Further, the chimeric protein may be an agonist or an antagonist of a third TGF-beta superfamily member.

In certain embodiments, a chimeric TGF-beta superfamily protein comprises core domains from at least two different naturally-occurring TGF-beta superfamily members. In certain embodiments, a chimeric TGF-beta superfamily protein comprises variable domains from at least two different naturally-occurring TGF-beta superfamily members. In certain embodiments one or more variable and/or core domains are randomized or otherwised altered so as not to correspond precisely to a variable domain of any naturally occurring TGF-beta superfamily protein. A core domain and/or a variable domain of a chimeric TGF-beta superfamily protein may comprise an amino acid addition, deletion, or substitution, or a modified amino acid.

A chimeric protein may comprise one or more post-translational modifications. Such modifications may be obtained by altering the sequence so as to provide a consensus amino acid sequence for post-translational modification. Alternatively, a chimeric protein may be derivatized, e.g. chemically or enzymatically, with the translational modification, with or without resort to any consensus amino acid sequence. Preferably a post-translational modification is positioned in a core domain, and a consensus amino acid sequence may be within a core domain of the chimeric protein. The post-translational modification may include, but is not limited to, glycosylation, phosphorylation, PEGylation, farnesylation, acetylation, biotinylation, lipidation (amino acid conjugated with lipid), conjugation with an organic derivatizing agent. The post-translational modification may improve stability, solubility, bioavailability, or biodistribution of the chimeric protein.

In certain embodiments, the subject chimeric protein binds to at least one of TGF-beta type 1 receptor and TGF-beta type 2 receptor. The chimeric protein may form a homodimer.

In preferred embodiments, the subject chimeric protein comprises a core domain derived from a Nodal polypeptide. The core domain may be derived from a murine or human Nodal polypeptide. The subject chimeric protein may comprise one, two, three, or four different core domains from a Nodal polypeptide. The subject chimeric protein may comprise a core domain comprising a sequence of SEQ ID NO: 2, a core domain comprising a sequence of SEQ ID NO: 3, a core domain comprises a sequence of SEQ ID NO: 4, and/or a core domain comprising a sequence of SEQ ID NO: 5.

In preferred embodiments, the subject chimeric protein comprises a sequence of any of SEQ ID NOs:10-31. The disclosure also provides proteins having sequences at least 80%, 85%, 90%, 95%, 97%, 98%, 99% or 100% identical to SEQ ID NO:10-31.

Modified proteins disclosed herein, including chimeric proteins, may be fused with one or more other proteins to create fusion proteins. The other proteins may include, but are not limited to, epitope tags (e.g., FLAG), purification tags (e.g., GST, (His)6)), stabilizers (e.g., Fc of an immunoglobulin, human serum albumin). Such fusion proteins may be easily purified and/or have enhanced stability.

A subject modified protein may also be used to screening for compounds that can modulate activities of the subject modified protein or a naturally-occurring TGF-beta superfamily member.

Also provided are nucleic acids encoding the subject modified proteins, including the chimeric proteins and fusion proteins. In preferred embodiments, a nucleic acid of the disclosure encodes a chimeric protein that comprises a sequence of any of SEQ ID NO:10-31. Methods of making the nucleic acids are also provided.

The disclosure further provides a recombinant polynucleotide comprising a promoter sequence operably linked to a nucleic acid encoding a subject chimeric protein. The recombinant polynucleotide may be employed to transform a host such as a cell. Cell transformed with the recombinant polynucleotide may be employed to express the subject modified protein, which can then be isolated or subject to assays.

The disclosure also provides a pharmaceutical preparation comprising a subject modified protein and a pharmaceutically acceptable carrier. A pharmaceutical preparation may be employed to promote growth of a tissue or diminishing or prevent loss of a tissue in a subject, preferably a human. The targeted tissue can be, for example, bone, cartilage, skeletal muscle, cardiac muscle and/or neuronal tissue.

Modified proteins of the disclosure may also be used in the manufacture of a medicament that can treat a condition such as for example a bone-related condition (e.g., osteoporosis), a skeletal muscle-related condition (e.g., a muscle wasting disease), a neurodegenative disease (such as Alzheimer's Disease) or a heart disease.

Further provided are methods of treatment. A method for treating a subject, preferably human, comprises administering to the subject an effective amount of a modified TGF-beta superfamily protein. The subject may have a disorder associated with insufficient bone mineral density, bone loss, bone damage, and/or insufficient bone growth. The subject may have lower than normal bone mineral density, osteoporosis, and/or a fracture. The subject may have a condition induced by excessive bone density and/or growth. Other subjects may have a disorder associated with abnormal amount, development or metabolic activity of muscle tissue, a muscle wasting disorder, cachexia, anorexia, Duchenne Muscular Dystrophy syndrome, Becker Muscular Dystrophy syndrome, AIDS wasting syndrome, muscular dystrophies, neuromuscular diseases, motor neuron diseases, diseases of the neuromuscular junction, and/or inflammatory myopathies. A subject may suffer from neurodegeneration such as for example Alzheimer's Disease, Parkinson's Disease (PD), Amyotrophic Lateral Sclerosis, or Huntington's disease. A subject may have a disorder associated with abnormal cell growth and differentiation which may cause inflammation, allergy, autoimmune diseases, infectious diseases, and/or tumors. A subject may have a heart disorder, such as a disorder associated with excessive cardiomyocyte proliferation or growth, or a disorder in which it would be desirable to stimulate cardiomyocyte growth or proliferation. Subject modified TGF-beta superfamily proteins may be designed for the treatment of essentially any disorder that is amenable to treatment by agonists or antagonists of a member of the TGF-beta superfamily.

The disclosure also provides a method for modulating the amount of a tissue, e.g., increasing growth of a tissue or decreasing loss of a tissue in a subject, comprising administering to the subject a sufficient amount of a modified TGF-beta superfamily protein.

### BRIEF DESCRIPTION OF THE DRAWINGS

FIG. 1 is a diagram showing a domain structure of an exemplary chimeric TGF-beta superfamily protein.

FIG. 2 shows a comparison of the three-dimensional structures for six TGF-beta superfamily members. Variable domains can be seen as those segments where the tertiary structures diverge.

FIG. 3 shows alignment of various TGF-beta superfamily member proteins, and the core and variable domains therein.

### DETAILED DESCRIPTION OF THE DISCLOSURE

### 1. Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

As used herein and in the appended claims, the singular forms “a,” “an,” and “the” include plural reference unless the context clearly dictates otherwise. Thus, for example, a reference to “a core domain” includes a plurality of core domains, and a reference to “an antibody” is a reference to one or more antibodies and equivalents thereof known to those skilled in the art, and so forth.

Variants of the amino acid sequence of the proteins of the present application include but are not limited to naturally occurring mature forms of the peptide, allelic/sequence variants of the peptides, non-naturally occurring recombinantly derived variants of the peptides, and orthologs and paralogs of the peptides.

As used herein, TGF-beta superfamily member refers to a TGF-beta superfamily (including bone morphogenic factors) gene or protein of any species, particularly a mammalian species, including but not limited to bovine, ovine, porcine, murine, equine, and human. “TGF-beta superfamily polypeptide” refers to the amino acid sequences of purified TGF-beta superfamily protein obtained from any species, particularly a mammalian species, including bovine, ovine, porcine, murine, equine, and human and from any source, whether natural, synthetic, semi-synthetic, or recombinant.

Each of these types of changes may occur alone, or in combination with the others, one or more times in a given sequence.

The term “aberrant” or “abnormal” process refers to a process that is altered, modified, or different from the normal physiological process occurring in a host cell.

“Altered” nucleic acid sequences encoding a polypeptide of the application include those sequences with deletions, insertions, or substitutions of different nucleotides, resulting in a polypeptide the same as the polypeptide of the application or a polypeptide with at least one functional characteristic thereof. Included within this definition are polymorphisms which may or may not be readily detectable using a particular oligonucleotide probe of the polynucleotide encoding a TGF-beta superfamily protein.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The term “biological activity” refers to a structural, regulatory, or biochemical function of a naturally occurring molecule.

The terms “bone loss” and “bone growth” are used herein to refer to changes (decreases or increases, respectively) in size or density of bone measured in any way, such as changes in bone volume, density or mineralization. For example, these characteristics may be assessed in terms of rates of loss or growth or in terms of snapshot or equilibrium comparisons.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies,” “intergenic,” etc. fusion of protein structures expressed by different kinds of organisms. “Chimeric TGF-beta protein,” “chimeric TGF-beta superfamily protein,” and “subject chimeric protein” are used interchangeably herein.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:

(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure or performs a particular function.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position, when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present application. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present application may be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the application. BLAST protein searches can be performed with the XBLAST program, score=50, wordlength=3 to obtain amino acid sequences homologous to protein molecules of the application. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988, Biocomputing: Informatics and Genome Projects, Smith, D. W., ed., Academic Press, New York, 1993, Computer Analysis of Sequence Data, Part I, Griffin, A. M., and Griffin, H. G., eds., Humana Press, New Jersey, 1994, Sequence Analysis in Molecular Biology, von Heinje, G., Academic Press, 1987, and Sequence Analysis Primer, Gribskov, M. and Devereux, J., eds., M Stockton Press, New York, 1991, and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894, Altschul, S., et al., J. Mol. Biol. 215: 403-410 (1990). The well known Smith Waterman algorithm may also be used to determine identity.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

“Protein,” “peptide,” and “polypeptide” are used interchangeably in this application.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified,” it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 6000 dalton, can be present). The term “pure” as used herein preferably has the same numerical limits as “purified” immediately above.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present application which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

“Small molecule” as used herein, is meant to refer to a composition, which has a molecular weight of less than about 6 kD and most preferably less than about 2.5 kD. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J. Am. Chem. Soc. 120, 8565-8566, 1998). It is within the scope of this application that such a library may be used to screen for agents of the disclosure.

There are numerous commercially available compound libraries, such as the Chembridge DIVERSet. Libraries are also available from academic investigators, such as the Diversity set from the NCI developmental therapeutics program. Rational drug design may also employed. For example, the interaction interface of a protein complex of the application may be targeted when designing a compound.

Peptidomimetics are compounds in which at least a portion of a subject polypeptide of the application is modified, and the three dimensional structure of the peptidomimetic remains substantially the same as that of the polypeptide. A subject polypeptide of the application may be a subject chimeric protein or a core or variable domain thereof. Peptidomimetics may be analogues of a subject polypeptide of the disclosure that are, themselves, polypeptides containing one or more substitutions or other modifications within the subject protein sequence. Alternatively, at least a portion of the subject polypeptide sequence may be replaced with a nonpeptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry. Each peptidomimetic may further have one or more unique additional binding elements. The present application provides methods for identifying peptidomimetics.

### 2. Overview

The proteins of the TGF-beta superfamily are usually disulfide-linked homo- or hetero-dimers that are expressed as large precursor polypeptides containing a hydrophobic signal sequence, a long and relatively poorly conserved N-terminal propeptide region sequence of several hundred amino acids, a cleavage site, a mature domain comprising an N-terminal region that varies among the family members and a highly conserved C-terminal region. This C-terminal region, present in the processed mature proteins of all known family members, contains approximately 100 amino acids with a characteristic cysteine motif having a conserved six or seven cysteine skeleton. Although the position of the cleavage site between the mature and propeptide regions varies among the family members, the cysteine pattern of the C-terminus of all of the proteins is in the identical format, ending in the sequence Cys-X-Cys-X (Sporn and Roberts (1990), supra).

A unifying feature of the biology of the proteins of the TGF-beta superfamily is their ability to regulate developmental processes. These structurally related member proteins have been identified as being involved in a variety of developmental events. Certain members of this same family of proteins are also morphogenic, i.e., capable of inducing the developmental cascade of tissue morphogenesis in a mature mammal (See PCT Application No. US 92/01968). In particular, these morphogens are capable of inducing the proliferation of uncommitted progenitor cells, and inducing the differentiation of these stimulated progenitor cells in a tissue-specific mariner under appropriate environmental conditions. In addition, the morphogens are capable of supporting the growth and maintenance of these differentiated cells. These morphogenic activities allow the proteins to initiate and maintain the developmental cascade of tissue morphogenesis in an appropriate, morphogenically permissive environment, stimulating stem cells to proliferate and differentiate in a tissue-specific manner, and inducing the progression of events that culminate in new tissue formation. These morphogenic activities also allow the proteins to induce the “redifferentiation” of cells previously stimulated to stray from their differentiation path. Under appropriate environmental conditions it is anticipated that these morphogens also may stimulate the “redifferentiation” of committed cells.

Scientists in the field have recognized five distinct forms of TGF-beta (TGF-β1-β5) as well as the differentiation factors (e.g., Vg-1), the hormones activin and inhibin, the Mullerianinhibiting substance (MIS), osteogenic and morphogenic proteins (e.g., OP-1, OP-2, OP-3, other BMPs), the developmentally regulated protein Vgr-1, the growth/differentiation factors (e.g., GDF-1, GDF-3, GDF-9 and dorsalin-1), etc. See, e.g., Spom and Roberts (1990) in Peptide Growth Factors and Their Receptors, Sporn and Roberts, eds., Springer-Verlag: Berlin pp. 419-472; Weeks and Melton (1987) Cell 51: 861-867; Padgett et al. (1987) Nature 325: 81-84; Mason et al. (1985) Nature 318: 659-663; Mason et al. (1987) Growth Factors 1: 77-88; Cate et al. (1986) Cell 45: 685-698; PCT/US90/05903; PCT/US91/07654; PCT/WO94/10202; U.S. Pat. Nos. 4,877,864; 5,141,905; 5,013,649; 5,116,738; 5,108,922; 5,106,748; and 5,155,058; Lyons et al. (1989) Proc. Natl. Acad. Sci. USA 86: 4554-58; McPherron et al. (1993) J. Biol. Chem. 268: 3444-3449; Basler et al. (1993) Cell 73: 687-702.

Morphogenic proteins of the TGF-beta superfamily include the mammalian osteogenic protein-1 (OP-1, also known as BMP-7), osteogenic protein-2 (OP-2, also known as BMP-8), osteogenic protein-3 (OP3), BMP-2 (also known as BMP-2A or CBMP-2A, and the Drosophila homolog DPP), BMP-3, BMP-4 (also known as BMP-2B or CBMP-2B), BMP-5, BMP-6 and its murine homolog Vgr-1, BMP-9, BMP-10, BMP-11, BMP-12, GDF3 (also known as Vgr2), GDF-8, GDF-9, GDF-10, GDF-11, GDF-12, BMP-13, BMP-14, BMP-15, GDF-5 (also known as CDMP-1 or MP52), GDF-6 (also known as CDMP-2 or BMP13), GDF-7 (also known as CDMP-3 or BMP-12), the Xenopus homolog Vgl and NODAL, UNIVIN, SCREW, ADMP, NEURAL, etc.

This disclosure provides core and variable domains of TGF-beta superfamily proteins. Core domains and variable domains may be interchanged between TGF-beta superfamily proteins to create chimeric proteins, which may be novel ligands of the TGF-beta receptors. The chimeric proteins are readily designed based on the naturally-occurring TGF-beta superfamily members and comprised of one or more domains derived from one or more TGF-superfamily member protein or gene. Core domains in particular are also amenable to post-translational modification, and accordingly the disclosure provides both chimeric and non-chimeric TGF-superfamily proteins having one or more post-translational modifications situated in a core domain.

The modified proteins may have properties that are more advantageous than naturally-occurring TGF-beta superfamily members. For example, such proteins may have desired structural or functional characteristics, such as enhanced or decreased binding to the TGF-beta receptors, enhanced stability compared to a naturally-occurring TGF-beta superfamily protein, agonistic (e.g., activating receptor-mediated signaling), or antagonistic (e.g., inhibiting receptor-mediated signaling) activity. A subject modified protein may be made in large quantity by recombinant technology. A modified protein may be more suitable than a naturally-occurring TGF-beta superfamily member for screening and identifying compounds that may modulate activities of the modified proteins and/or the naturally-occurring TGF-beta superfamily member.

### 3. Core and Variable Domains and Modified Proteins

Accordingly, the disclosure provides modified TGF-beta superfamily proteins.

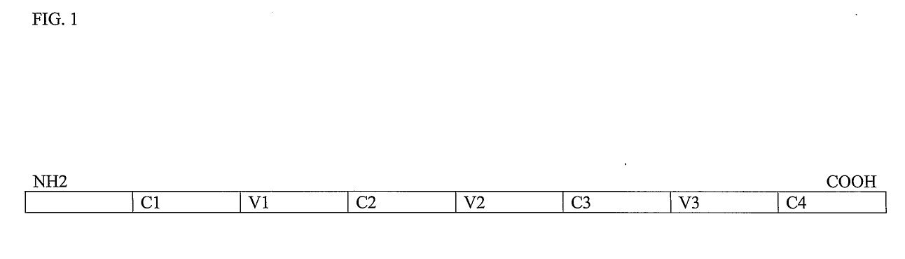

In certain embodiments, the disclosure provides chimeric proteins comprising at least one core domain from a first TGF-beta superfamily protein and at least one variable domain from a second TGF-beta superfamily protein. Note that core and variable domains may be altered with respect to the amino acid sequence that is naturally found in either the first or second TGF-beta superfamily protein. In certain embodiments, a subject chimeric protein comprises four core domains (e.g., C1-C4) from a first TGF-beta superfamily protein interposed by three variable domains (e.g., V1-V3) from a second TGF-beta superfamily protein. By “interposed” is meant a sequence alignment of the core and variable domains such as C1-V1-C2-V2-C3-V3-C4 (See, e.g., FIG. 1). In certain embodiments, the subject chimeric protein comprises variable domains from at least two different naturally-occurring TGF-beta superfamily proteins. In certain embodiments, the subject chimeric protein comprises core domains from at least two different naturally-occurring TGF-beta superfamily proteins.

a. Core Domains and Variable Domains

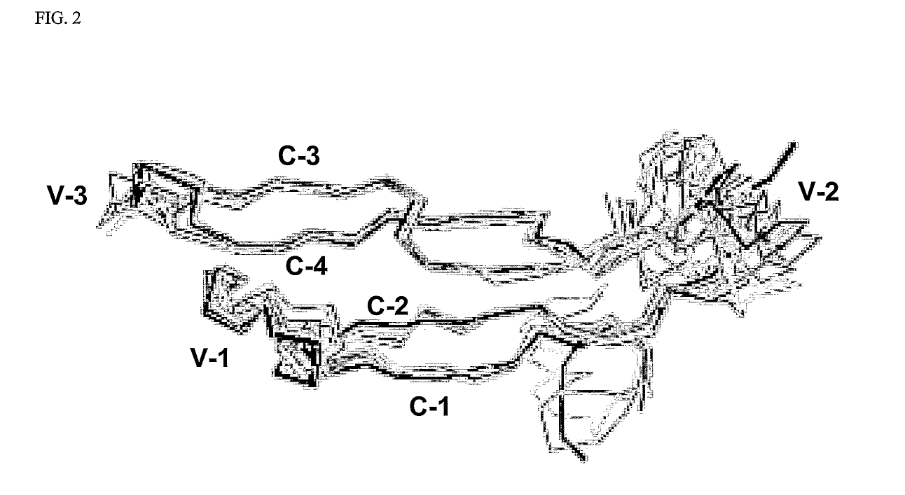

Core domains and variable domains from various TGF-beta superfamily members can be identified by different methods. In preferred embodiments, such core domains and/or variable domains may be identified based on the member proteins' tertiary structures, e.g., as shown in FIG. 2.

FIG. 2 shows positions of the respective core domains C1-C4 and the variable domains V1-V3 on the superposed tertiary structures of TGF-beta 2, TGF-beta 3, BMP-2, and BMP-7. Thompson et al., EMBO J. 22(7):1555-1566 (2003).

For other TGF-beta superfamily members of which the tertiary structures are yet to be resolved, structure modeling methodologies may be employed to predict their tertiary structures. The predicted tertiary structures can then be employed to determine the respective core domains and variable domains. One methodology of protein tertiary structure modeling is termed “homology modeling,” which employs a suitable known structure as a starting point. Homology modeling may be preferred because of high percentage of homology shared among TGF-beta superfamily members, especially within a subfamily or subgroup of the member proteins, e.g., BMPs or GDFs, TGF-betas, or activins. Generally, homology modeling involves the following:

1) Finding a suitable starting model: Homology modeling generally depends on the correctness of the assumption that the proteins are homologous and that the protein of unknown structure has the same general fold as the protein of a known structure. Usually, a known structure is chosen based on the highest degree of sequence similarity between the proteins, but it would be useful to include information from more than one known structure in the modeling, e.g., any of the structures or combinations thereof as shown in FIG. 2 may be used.

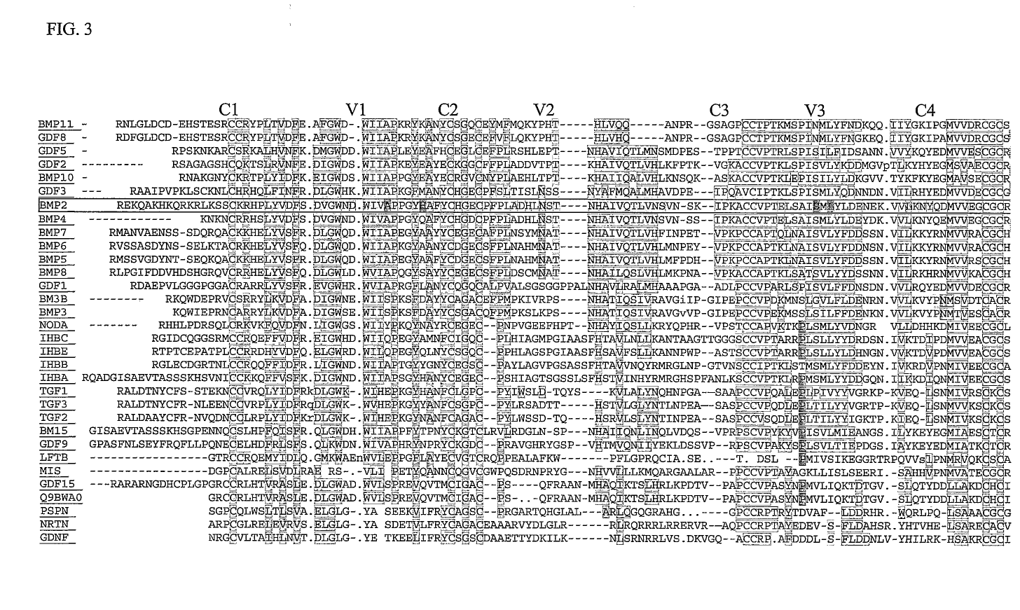

2) Alignment: When the sequence similarity is high, the alignment is readily achieved, e.g., the alignment as shown in FIG. 3. For distantly related proteins, alignments based on many sequences using methods based on Hidden Markov Models may prove more useful than pairwise alignments

3) Modeling: Usually, the secondary structure elements of the known protein are used as the starting model, but depending on the degree of similarity, loop regions can also be included. For modeling of loops of unknown conformation, a database of observed loop conformations can be used. The actual modeling can be done very simply by replacing amino acids. In a suitable graphics program, manual modifications can be done to avoid obvious problems with for example colliding side chains. This modeling can be complemented with energy minimization or other refinement protocols. Since the starting model is based on experiment and is relatively accurate, the model in those core regions where only small changes are predicted might be left without refinement.

Homology modeling can result in fairly accurate models, especially in cases where the starting model has a high degree of sequence similarity to the unknown protein. The quality of a model will vary between the regions in the core of the protein and the loop regions. The conformation of surface loops can be expected to have a more different conformation, and some procedures avoid modeling these loops. When the sequence similarity is low (below 30%), models based on sequence homology will most likely be partly incorrect. Even if the fold is correct, the difficulties in aligning sequences correctly make it likely that the sequence will be fitted incorrectly not only in surface loops, but possibly also in secondary structure elements.

Several programs are available for homology modeling. A server which offers homology modeling from a sequence is SwissModel. In this server, the procedure described above is followed. In the first step, a number of suitable known structures with significant sequence similarity to the search-sequence are found using a BLAST search of a database of known structures. In the second step, the sequences are aligned. At both these stages, the user might interact with the server and choose template structures or adjust the sequence alignment. The model is constructed, and in the final step, an energy minimization using the GROMOS96 potentials is performed.

An important feature is that a quality estimate is attached to every atom of the model. This “model confidence factor” is based on the number of template models, the similarity of template models themselves and similarity of the model to the template model(s). In this way, an observed conformational variability is taken into account when the accuracy of the model is estimated.

The Modeller is another program for homology modeling. This program is performing only the model building, and the user has to supply the alignment of the search sequence to the template model(s).

Homology modeling may also employ threading method. A threading method attempts to fit the sequence to a fold. Mimy and Shakhnovich (J Mol. Biol. 1998 Oct. 23; 283(2):507-26) described a novel Monte Carlo threading algorithm which allows gaps and insertions both in the template structure and threaded sequence. The algorithm is able to find the optimal sequence-structure alignment and sample suboptimal alignments. The computer system PROSPECT for the protein fold recognition using the threading method is analyzed in Xu and Xu, Proteins. 2000 Aug. 15; 40(3):343-54.

Molecular structure modeling may also be carried out using similar programs, materials, and methods as described in Sheppard et al., Functional and Structural Diversity in the Als Protein Family of Candida albicans, J Biol. Chem. (May 5, 2004).

Core domains and variable domains of certain TGF-beta superfamily members may also be identified based on the alignment provided in FIG. 3. The approximate regions of the aligned sequences that correspond to C1-C4 and V1-V3 are shown in this figure.

A core domain or variable domain of a TGF-beta superfamily member may be identified based on the tertiary structure of the member protein and/or the primary amino acid sequence as aligned against other homologous member proteins. As identified, a core domain or variable domain of the disclosure may comprise a particular amino acid sequence or an original amino acid sequence that is amenable to substitution(s), insertion(s), additional amino acid(s) at either or both termini of the original sequence, or other modifications. By “amenable” is meant that the structural integrity of the core domain or variable domain is maintained as compared to the domain of the original sequence. For example, a core domain or variable domain of a TGF-beta superfamily member may shift by 10, 5, 3, 2, or 1, or preferably no more than 1 amino acid on either or both termini of the core or variable domain as identified. For example, dvgwndwivappgyh represents a variable domain of BMP-2 as identified based on BMP-2's tertiary structure (referring to the human BMP-2 precursor sequence below). The variable domain may, for example shift by 1 amino acid at its N-terminus and comprise an amino acid sequence of sdvgwndwivappgyh or vgwndwivappgyh.

A core domain of the disclosure may be a naturally-occurring core domain of a TGF-beta superfamily member. Alternatively, a core domain of the disclosure may comprise a modified naturally-occurring core domain. The modification may comprise altered length of the domain, an amino acid addition or deletion, an amino acid modification (e.g., lipidation, phosphorylation), and/or an amino acid substitution, so long as structural integrity and/or functionality of the domain is maintained after the modification. The term “maintained” is not meant to indicate precise identity in structural features but merely sufficient similarity such that structural integrity and/or functionality is not completely disrupted, and preferably structural integrity and functionality are substantially retained.

Similarly, a variable domain of the disclosure may be a naturally-occurring variable domain of a TGF-beta superfamily member. Alternatively, a variable domain of the disclosure may comprise a modified naturally-occurring variable domain. The modification may comprise altered length of the domain, an amino acid addition or deletion, an amino acid modification (e.g., lipidation, phosphorylation), and/or an amino acid substitution, so long as structural integrity and/or functionality of the domain is maintained after the modification. A variable portion may also be completely or partially randomized. Libraries of modified TGF-beta polypeptides with variations in one or more variable domains may be screened to identify combinations of variable domain and core domain sequences that provide desirable characteristics.

To illustrate, the variable domains in BMP-2 are represented by the amino acids in bold letters in the following sequence:

gi|4557369|ref|NP_{—001191.1|bone morphogenetic}
protein 2 precursor [Homo sapiens]

| | |
|---|---|
| 1 | mvagtrclla lllpqvllgg aaglvpelgr rkfaaassgr |
| | pssqpsdevl sefelrllsm |
| 61 | fglkqrptps rdavvppyml dlyrrhsgqp gspapdhrle |
| | raasrantvr sfhheeslee |
| 121 | lpetsgkttr rfffnlssip teefitsael qvfregmqda |
| | lgnnssfhhr iniyeiikpa |
| 181 | tanskfpvtr lldtrlvnqn asrwesfdvt pavmrwtaqg |
| | hanhgfvvev ahleekqgvs |
| 241 | krhvrisrsl hqdehswsqi rpllvtfghd gkghplhkre |
| | krqakhkgrk rlkssckrhp |
| 301 | lyvdfsdvgw ndwivappgy hafychgecp fpladhlnst |
| | nhaivqtlvn svnskipkac |
| 361 | cvptelsais mlyldenekv vlknyqdmvv egcgcr |

In the BMP-2 precursor sequence above, the amino acid sequence of amino acid position 282-396 (underlined) represents the mature BMP-2 sequence. The three domains in bold letters represent preferred variable domains (V1-V3) of BMP-2. The four domains flanked by these three variable domains in the mature BMP-2 sequence (the underlined but not bold letters) represent preferred core domains (C1-C4) of BMP-2.

FIG. 3 further provides alignment of various TGF-beta superfamily members, and illustrates the core and variable domains therein.

Also provided are core domain and variable domain sequences in a Nodal protein, preferably a murine Nodal protein (e.g., SEQ ID NO:2-5 for core domains, and SEQ ID NO:6-8 for variable domains).

| | |
|---|---|
| SEQ ID NO:1 | RHHLPDRSQLCRRVKFQVDFNLIGWGSQIIY |
| (murine Nodal): | PKQNAYRCEGECPNPVGEEFHPTNHAYIQSL |
| | LKRYQPHRVPSTCCAPVKTKPLSMLYVDNGR |
| | VLLEHHKDMIVEECGCL |
| SEQ ID NO:2 (C1): | RHHLPDRSQLCRRVKFQVDFN |
| SEQ ID NO:3 (C2): | YRCEGEC |
| SEQ ID NO:4 (C3): | YQPHRVPSTCCAPVKTK |
| SEQ ID NO:5 (C4): | KDMIVEECGCL |
| SEQ ID NO:6 (V1): | LIGWGSWIIYPKQYNA |
| SEQ ID NO:7 (V2): | PNPVGEEFHPTNHAYIQSLLKR |
| SEQ ID NO:8 (V3): | PLSMLYVDNGRVLLEHH |

Core domains and variable domains of a human Nodal are also provided and can be found through aligning the human Nodal and murine Nodal amino acid sequences.

(human Nodal):

SEQ ID NO:9

RHHLPDRSQLCRKVKFQVDFNLIGWGSWIIYPKQYNAYRCEGECPNPVGE
EFHPTNHAYIQSLLKRYQPHRVPSTCCAPVKTKPLSMLYVDNGRVLLDHH
KDMIVEECGCL

### Alignment Human Nodal and Murine Nodal

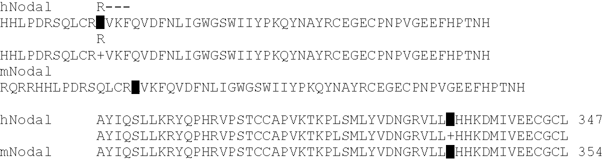

Accordingly, in certain embodiments the disclosure provides chimeric TGF-beta superfamily proteins comprising one or more core domain from a first naturally-occurring TGF-beta superfamily protein and one or more variable domain from a second naturally-occurring TGF-beta superfamily protein. While not wishing to be bound by a particular theory, the one or more variable domain of a subject chimeric protein contributes or defines receptor specificity of the chimeric protein and the one or more core domain of the chimeric protein provides a structural support for the chimeric protein (as a scaffold).

In particular embodiments, the disclosure provides chimeric TGF-beta superfamily proteins comprising an amino acid sequence of any of SEQ ID NO:10-31. The disclosure further provides proteins having sequences at least 80%, 85%, 90%, 95%, 97%, 98%, 99% or 100% identical to SEQ ID NO:10-31.

(mNodal-BMP-2):

SEQ ID NO:10

RHHLPDRSQLCRRVKFQVDFNDVGWNDWIVAPPGYHYRCEGECPFPLADH
LNSTNHAIVQTLVNSYQPHRVPSTCCAPVKTKSAISMLYLDENEKVVLKN
YKDMIVEECGGL
(mNodal-BMP-11):

SEQ ID NO:11

RHHLPDRSQLCRRVKFQVDFNAFGWDWIIAPKRYKYRCEGECPEFVFLQK
YPHTHLVQHYQPHRVPSTCCAPVKTKSPINMLYFNGKQQIIYGKIKDMIV
EECGCL
(mNodal-Activin):

SEQ ID NO:12

RHHLPDRSQLCRRVKFQVDFNDIGWNDWIIAPSGYHYRCEGECPSHIAGT
SGSSLSFIISTVINHYRYQPHRVPSTCCAPVKTKRPMSMLYYDDGQNIIK
KDIKDMIVEECGCL
(mNodal-GDF-3):

SEQ ID NO:13

RHHLPDRSQLCRRVKFQVDFNDLGWHKWIIAPCGFMYRCEGECPFSLTIS
LNSSNYAFMQALMHAYQPHRVPSTCCAPVKTKSPISMLYQDNNDNVILRH
YKDMIVEECGCL
(mNodal-BMP-10):

SEQ ID NO:14

RHHLPDRSQLCRRVKFQVDFNEIGWDSWIIAPPGYEYRCEGECYPLAEHL
TPTKHAIIQALVHLYQPHRVPSTCCAPVKTKEPISILYLDKGVVTYKFKY
KDMIVEECGCL
(mNodal-GDF-8):

SEQ ID NO:15

RHHLPDRSQLCRRVRFQVDFNAFGWDWIIAPKRYKYRCEGECEFVFLQKY
PHTHLVHQYQPHRVPSTCCAPVKTKSPINMLYFNGKEQIIYGKIKDMIVE
ECG
(mNodal-GDF-5):

SEQ ID NO:16

RHHLPDRSQLCRRVKFQVDFNDMGWDDWIIAOLEYEYRCEGECEFPLRSH
LEPTNHAVIQTLMNSYQPHRVPSTCCAPVKTKSPISILFIDSANNVVKQY
KDMIVEECGCL
(mNodal-GDF-6):

SEQ ID NO:17

RHHLPDRSQLCRRVKEQVDFNELGWDDWIIAPLEYEYRCEGEGDFPLRSH
LEPTNHAIIQTLMNSYQPHRVPSTCCAPVKTKTPISILYIDAGNNVVYNE
YKDMIVEECGCL
(mNodal-GDF-7):

SEQ ID NO:18

RHHLPDRSQLCRRVKFQVDFNELGWDDWIIAPLDYEYRCEGECDFPLRSH
LEPTNHAIIQTLLNSYQPHRVPSTCCAPVKTKSPISILYIDAANNVVYKQ
YKDMIVEECGCL
(mNodal-GDF-10):

SEQ ID NO:19

RHHLPDRSQLCRRVKFQVDFNEIGWDSWIIAPPGYEYRCEGECNYPLAEH
LTPTKHAIIQALVHLYQPHRVPSTCCAPVKTKEPISILYLDKGVVTYKFY
KDMIVEECGCL
(mNodal-BMP-4):

SEQ ID NO:20

RHHLPDRSQLCRRVKFQVDFNDVGWNPWIVAPPGYQYRCEGECPFPLADH
LNSTNHAIVQTLVNSYQPHRVPSTCCAPVKTKSAISMLYLDEYDKVVLKN
YKDMIVEECGGL
(mNodal-BMP-7):

SEQ ID NO:21

RHHLPDRSQLCRRVKFQVDFNDLGWQDWIIAPEGYAYRCEGECAFPLNSY
MNATNHAIVQTLVHFYQPHRVPSTCCAPVKTKNAISVLYFDDSSNVILKD
MIVEECGCL
(mNodal-BMP-6):

SEQ ID NO:22

RHHLPDRSQLCRRVKFQVDFNDLGWQDWIIAPKGYAYRCEGECSFPLNAH
MNATNHAIVQTLVHLYQPHRVPSTCCAPVKTKNAISVLYFDDNSNVILKK
YKDMIVEECGCL
(mNodal-BMP-5):

SEQ ID NO:23

RHHLPDRSQLCRRVKFQVDFNDLGWQDWIIAPEGYAYRCEGECSFPNLAH
MNATNHAIVQTLVHLYQPHRVPSTCCAPVKTKNAISVLYFDDSSNVILKD
MIVEECGCL
(mNodal-BMP-8):

SEQ ID NO:24

RHHLPDRSQLCRRVKFQVDFNDLGWLDWVIAPQGYSYRCEGECSFPLDSC
MNATNHAILQSLVHLYQPHRVPSTCCAPVKTKSATSVLYYDSSNNVILRK
HKDMIVEECGCL
(mNodal-GDF-1):

SEQ ID NO:25

RHHLPDRSQLCRRVKFQVDFNEVGWHRWVIAPRGFLYRCEGECALPVALS
GSGGPPALNHAVLRALMHAAYQPHRVPSTCCAPVKTKSPISVLFFDNSDN
VVLRQYKDMIVEECGCL
(mNodal-BMP-3):

SEQ ID NO:26

RHHLPDRSQLCRRVKFQVDFNDIGWSEWIISPKSFDYRCEGECQFPMPKF
LKPSNHATIQSIVRAYQPHRVPSTCCAPVKTKSSLSILFFDENKNVVLKV
YKDMIVEECGCL
(mNodal-TGF-beta 1):

SEQ ID NO:27

RHHLPDRSQLCRRVKEQVDFNDLGWKWIHTKGYHYRCEGECPYIWSLDTQ
YSKVLALYNQHYQPHRVPSTCCAPVKTKEPLPIVYYVGRKPKVEQLKDMI
VEECGCL
(mNodal-BMP-15):

SEQ ID NO:28

RHHLPDRSQLCRRVKFQVDFNQLGWDHWIIAPPFYTYRCEGECLRVLRDG
LNSPNHAIIQNLINQLYQPHRVPSTCCAPVKTKVPISVLMIEANGSILYK
EYKDMIVEEGGCL
(mNodal-GDF-9):

SEQ ID NO:29

RHHLPDRSQLCRRVKFQVDFNQLKWDNWIVAPHRYNYRGEGECPRAVGHR
YGSPVHTMVQNIIYEKYQPHRVPSTCCAPVKTKSPLSVLTIEPDGSIAYK
EYKDMIVEECGCL
(mNodal-GDF-15):

SEQ ID NO:30

RHHLPDRSQLCRRVKPQVDFNDLGWADWVLSPREVQYRCEGECPSQFRAA
NMHAQIKTSLHRLYQPHRVPSTCCAPVKTKNPMVLIQKTDTGVSLQTYKD
MIVEECGCL
(mNodal-LEFTY):

SEQ ID NO:31

RHHLPDRSQLCRRVKFQVDFNGMKAENWVLEPPGFLYRCEGECPRAVGHR
YGSPVHTMVQNIIYEKYQPHRVPSTCCAPVKTKSPLSVLTIEPDGSIAYK
EYKDMIVEECGCL

The underlined domains in SEQ ID NOS: 10-31 as shown above correspond to variable domains of the respective TGF-beta superfamily members as indicated (other than mNodal). Accordingly, core domains for each of these member proteins can be deduced by subtracting these variable domain sequences from the respective amino acid sequences of these member proteins. Also as described above, each of the core or variable domain may be amenable to substitution(s), insertion(s), additional amino acid(s) at either or both termini of the domain, or other modifications, so long as the structural integrity of the modified domain is maintained as compared to the domain with the original sequence, e.g., any of the underlined sequences above or any of SEQ ID NOS:2-8. Additionally, a core or variable domain may shift by 10, 5, 3, 2, or 1, or preferably no more than 1 amino acid on either or both termini of the core or variable domain as identified above, e.g., any of the underlined sequences above or any of SEQ ID NOS:2-8.

In certain embodiments, the disclosure provides TGF-beta superfamily proteins comprising one or more post-translational modifications. Such post-translational modifications are preferably situated in one or more core domains, but variable domains containing post-translational modifications are also contemplated.

TABLE 1
Examples of TGF-beta superfamily members known in the art.

| Name | Exemplary References |
|---|---|
| BMP-2 | Wozney et al. (1988) Science 242: (1528-1534 |
| BMP-3 | Wozney et al. (1988) Science 242: (1528-1534 |
| BMP-4 | Wozney et al. (1988) Science 242: (1528-1534 |
| BMP-5 | Celeste et al. (1990) Proc. Natl. Acad. Sci. |
| | USA. 87: 9843-9847 |
| BMP-6 | Celeste et al. (1990) Proc. Natl. Acad. Sci. |
| | USA. 87: 9843-9847 |
| BMP-7 | Celeste et al. (1990) Proc. Natl. Acad. Sci. |
| (OP-1) | USA. 87: 9843-9847 |
| BMP-8 | Ozkaynak et al. (1992) J. Biol. Chem. |
| (OP-2) | 267: 25220-25227 |
| BMP-10 | Neuhaus et al., Mech. Dev. 80 (2), |
| | 181-184 (1999) |
| BMP-15 | Dube et al. Molec. Endocr. 12: |
| (GDF-9B) | 1809-1817, 1998. |
| OP-3 | Ozkaynak et al. PCT/WO94/10203 SEQ ID NO: 1 |
| GDF-1 | Lee (1990) Mol. Endocrinol. 4: 1034-1040 |
| GDF-3 | Caricasole et al., Oncogene 16: 95-103, 1998; |
| | McPherron et al. (1993) J. Biol. Chem. |
| | 268: 3444-3449 |
| GDF-5 | Hotten et al., Biochem Biophys Res Commun. |
| (CDMP-1) | 1994 Oct 28; 204(2): 646-52. |
| GDF-6 | Storm et al., Nature. 1994 Apr 14; |
| (BMP-13) | 368(6472): 639-43. |
| GDF-7 | Storm et al., Nature. 1994 Apr 14; |
| (BMP-12) | 368(6472): 639-43. |
| GDF-8 | McPherron et al., Nature. 1997 May 1; |
| | 387(6628): 83-90. |
| GDF-9 | McGrath et al., Molec. Endocr. 9: 131-136, 1995; |
| | McPherron et al. (1993) J. Biol. Chem. 268: |
| | 3444-3449 |
| GDF-10 | Hino et al. Biochem. Biophys. Res. Commun. |
| (BMP-3B) | 223: 304-310, 1996. |
| GDF-11 | Nakashima et al., Mech Dev. 1999 Feb; |
| (BMP-11) | 80(2): 185-9. |
| GDF-15 | Bootcov et al., Proc Natl Acad Sci U S A. 1997 Oct |
| (MIC-1) | 14; 94(21): 11514-9. |
| Lefty | Kosaki et al., Am J Hum Genet. 1999 Mar; 64(3): 712-21. |
| Inhibin | Forage et al. (1986) Proc Natl. Acad. Sci. USA 83: |
| βA | 3091-3095; Chertov et al.(1990) Biomed |
| (Activin | Sci. 1: 499-506 |
| A) | |
| Inhibin | Mason et al. (1986) Biochem. Biophys. |
| βB | Res. Commun. 135: 957-964. |
| (Activin | |
| B) | |
| Inhibin α | Mayo et al. (1986) Proc. Natl.. Acad. Sci. USA 83 |
| | 5849-5 853. |
| TGF-β1 | Derynck et al. (1987) Nuci. Acids. Res. 15; 3187 |
| TGF-β2 | Burt et al. (1991) DNA Cell Biol. 10: 723-734 |
| TGF-β3 | ten Dijke et al. (1988) Proc. Natl. Acad. Sci. |
| | USA 85: 4715-4719; |
| | Derynck et al. (1988) EMBO J.7: 3737-3743 |
| TGF-β4 | Burt et al. (1992) Mol. Endcrinol. 6: 989-922 |
| TGF-β5 | Kondaiah et al. (1990) J. Biol. Chem. 265: |
| | 1089-1093 |

In certain embodiments, the present disclosure makes available isolated and/or purified forms of the modified TGF-beta superfamily proteins, which are isolated from, or otherwise substantially free of, other proteins.

In certain embodiments, a modified TGF-beta superfamily protein (chimeric and/or post-translationally modified) of the disclosure can be produced by a variety of art-known techniques as described in more detail below. For example, a modified TGF-beta superfamily protein can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, modified TGF-beta superfamily proteins, fraginents or variants thereof may be recombinantly produced using various expression systems (e.g., E. coli, Chinese Hamster Ovary cells, COS cells, baculovirus) as is well known in the art (also see below).

b. Variant Chimeric Proteins

In certain embodiments, the present disclosure contemplates making functional variants by altering the structure of a modified TGF-beta superfamily protein for purposes such as for example enhancing therapeutic efficacy, or stability (e.g., ex vivo shelf life and resistance to proteolytic degradation in vivo). Such modified TGF-beta superfamily proteins when designed to retain at least one activity of the naturally-occurring form of a TGF-beta superfamily protein with which a common variable domain is shared, are considered functional equivalents of the naturally-occurring TGF-beta superfamily protein. Modified chimeric TGF-beta superfamily proteins can also be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a chimeric TGF-beta superfamily protein results in a functional homolog can be readily determined by assessing the ability of the variant chimeric protein to produce a response in cells in a fashion similar to its respective original chimeric protein.

In certain embodiments, the present disclosure contemplates specific mutations of the chimeric or non-chimeric TGF-beta superfamily protein sequences, e.g., of a core domain sequence, so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (where “X” is any amino acid) which are specifically recognized by appropriate cellular glycosylation enzymes. The alteration may also be made by the addition of, or substitution by, one or more serine or threonine residues to the sequence of the wild-type chimeric TGF-beta superfamily protein (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a chimeric or non-chimeric TGF-beta superfamily protein is by chemical or enzymatic coupling of glycosides to the TGF-beta superfamily protein. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on a chimeric TGF-beta superfamily protein may be accomplished chemically and/or enzymatically. Chemical deglycosylation may involve, for example, exposure of the chimeric TGF-beta superfamily protein to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on chimeric TGF-beta superfamily proteins can be achieved by the use of a variety of endo- and exo-glycosidases as described by Thotakura et al. (1987) Meth. Enzymol. 138:350. The sequence of a modified chimeric or non-chimeric protein may be adjusted, as appropriate, depending on the type of expression system used, as mammalian, yeast, insect and plant cells may all introduce differing glycosylation patterns that can be affected by the amino acid sequence of the peptide.

This disclosure further contemplates a method of generating mutants, particularly sets of combinatorial mutants of the chimeric TGF-beta superfamily protein, as well as truncation mutants; pools of combinatorial mutants are especially useful for identifying functional variant sequences. The purpose of screening such combinatorial libraries may be to generate, for example, chimeric TGF-beta superfamily protein variants which can act as either agonists or antagonist, or alternatively, which possess novel activities all together. A variety of screening assays are provided below, and such assays may be used to evaluate variants. For example, a chimeric TGF-beta superfamily protein variant may be screened for ability to bind to a mature TGF-beta superfamily polypeptide or a TGF-beta receptor, or for the ability to prevent binding of a mature TGF-beta superfamily polypeptide to a cell expressing a TGF-beta receptor, such as an ActRII. The activity of a subject chimeric protein may also be tested in a cell-based or in vivo assay. For example, the effect of a chimeric TGF-beta superfamily protein on BMP-2-induced expression of genes involved in bone production in an osteoblast or precursor may be assessed. This may, as needed, be performed in the presence of recombinant BMP-2, and cells may be transfected so as to produce any of BMP-2 and the subject chimeric TGF-beta superfamily protein variant. Likewise, a chimeric TGF-beta superfamily protein may be administered to a mouse or other animal, and one or more bone properties, such as density or volume may be assessed. The healing rate for bone fractures may also be evaluated.

Combinatorially-derived variants can be generated which have a selective potency relative to a chimeric TGF-beta superfamily protein. Such variant proteins, when expressed from recombinant DNA constructs, can be used in gene therapy protocols. Likewise, mutagenesis can give rise to variants which have intracellular half-lives dramatically different than the corresponding original chimeric protein. For example, the altered protein can be rendered either more stable or less stable to proteolytic degradation or other cellular process which result in destruction of, or otherwise inactivation of a native chimeric TGF-beta superfamily protein. Such variants, and the genes which encode them, can be utilized to alter chimeric TGF-beta superfamily protein levels by modulating the half-life of the chimeric protein. For instance, a short half-life can give rise to more transient biological effects and, when part of an inducible expression system, can allow tighter control of recombinant chimeric TGF-beta superfamily protein levels within the cell.

In a preferred embodiment, the combinatorial library is produced by way of a degenerate library of genes encoding a library of polypeptides which each include at least a portion of potential chimeric TGF-beta superfamily protein sequences. For instance, a mixture of synthetic oligonucleotides can be enzymatically ligated into gene sequences such that the degenerate set of potential chimeric TGF-beta superfamily protein nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, chimeric TGF-beta superfamily protein variants (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell. Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of chimeric TGF-beta superfamily proteins.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of chimeric TGF-beta superfamily proteins. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, the chimeric TGF-beta superfamily proteins of the present disclosure include peptidomimetics. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the chimeric TGF-beta superfamily proteins.

To illustrate, by employing scanning mutagenesis to map the amino acid residues of a chimeric TGF-beta superfamily protein which are involved in binding to another protein, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

In certain embodiments, the modified chimeric or non-chimeric TGF-beta superfamily proteins of the disclosure may further comprise post-translational modifications in addition to any that are naturally present in the modified protein. For example, a chimeric or non-chimeric protein of the disclosure may comprise glycosylation or other modification in one or more of its core domains. Such modifications include, but are not limited to, acetylation, carboxylation, glycosylation, phosphorylation, lipidation, and acylation. As a result, the modified TGF-beta superfamily proteins may contain non-amino acid elements, such as polyethylene glycols, lipids, poly- or mono-saccharide, and phosphates. Effects of such non-amino acid elements on the functionality of a modified TGF-beta superfamily protein may be tested as described herein for other TGF-beta superfamily protein variants. When a modified TGF-beta superfamily protein is produced in cells by cleaving a nascent form of the precursor protein, post-translational processing may also be important for correct folding and/or function of the protein. Different cells (such as CHO, HeLa, MDCK, 293, W138, NIH-3T3 or HEK293) have specific cellular machinery and characteristic mechanisms for such post-translational activities and may be chosen to ensure the correct modification and processing of the precursor protein into a modified TGF-beta superfamily protein.

In certain aspects, functional variants of the chimeric or non-chimeric TGF-beta superfamily proteins include fusion proteins having at least a portion of the chimeric TGF-beta superfamily proteins and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of a subject chimeric protein. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-lnyc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, a modified TGF-beta superfamily protein is fused with a domain that stabilizes the chimeric protein in vivo (a “stabilizer” domain, e.g., a human serum albumin). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties such as enhanced stability. Other types of fusion domains that may be selected include multimerizing (e.g., dimerizing, tetramerizing) domains and functional domains (that confer an additional biological function, such as further stimulation of bone growth).

It is understood that different elements of the fusion proteins may be arranged in any manner that is consistent with the desired functionality. For example, a modified TGF-beta superfamily protein may be placed C-terminal to a heterologous domain, or, alternatively, a heterologous domain may be placed C-terminal to a modified TGF-beta superfamily protein. The modified protein and the heterologous domain need not be adjacent in a fusion protein, and additional domains or amino acid sequences may be included C- or N-terminal to either domain or between the domains.

In certain embodiments, the modified TGF-beta superfamily proteins of the present disclosure contain one or more alterations that are capable of stabilizing the chimeric TGF-beta superfamily proteins. For example, such alterations may enhance the in vitro half life of the modified proteins, enhance circulatory half life of the chimeric proteins or reducing proteolytic degradation of the chimeric proteins. Such stabilizing alterations include, but are not limited to, fusion proteins (including, for example, fusion proteins comprising a chimeric TGF-beta superfamily protein and a stabilizer domain), alterations of a glycosylation site (including, for example, addition of a glycosylation site to a chimeric TGF-beta superfamily protein, e.g., in a core domain), and alterations of carbohydrate moiety (including, for example, removal of carbohydrate moieties from a chimeric TGF-beta superfamily protein). In the case of fusion proteins, a chimeric TGF-beta superfamily protein is fused to a stabilizer domain such as an IgG molecule (e.g., an Fc domain) or human serum albumin. As used herein, the term “stabilizer domain” not only refers to a fusion domain (e.g., Fc, or serum albumin) as in the case of fusion proteins, but also includes nonproteinaceous alterations such as a carbohydrate moiety, or nonproteinaceous polymer, such as polyethylene glycol.

### 4. Nucleic Acids of the Disclosure and Uses Thereof

The disclosure further provides nucleic acids relating to the subject modified chimeric and non-chimeric TGF-beta superfamily proteins.

In certain aspects, the disclosure provides isolated and/or recombinant nucleic acids encoding any of the modified TGF-beta superfamily proteins, including functional variants, disclosed herein. An exemplary chimeric TGF-beta superfamily protein comprises a sequence of any of SEQ ID NO:10-31. The subject nucleic acids may be single-stranded or double stranded. Such nucleic acids may be DNA or RNA molecules. These nucleic acids are may be used, for example, in methods for making chimeric TGF-beta superfamily proteins or as direct therapeutic agents (e.g., in a gene therapy approach).

In certain embodiments, the disclosure provides isolated or recombinant nucleic acid sequences that encode protein sequences at least 80%, 85%, 90%, 95%, 97%, 98%, 99% or 100% identical to SEQ ID NO:10-31. In further embodiments, the nucleic acid sequences of the disclosure can be isolated, recombinant, and/or fused with a heterologous nucleotide sequence, or in a DNA library.

A nucleic acid encoding a chimeric protein of the disclosure comprises a fusion gene, methods of making which are known in the art. For example, the joining of various DNA or gene fragments (e.g., a DNA fragment comprising a nucleic acid encoding a core domain from a first TGF-beta superfamily protein, and a DNA fragment comprising a nucleic acid encoding a variable domain from a second TGF-beta superfamily protein) coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In certain embodiments, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

As mentioned above, the recombinant nucleic acid constructs of the disclosure can be manufactured by using conventional recombinant DNA methodologies well known and thoroughly documented in the art, as well as by using well-known biosynthetic and chemosynthetic methodologies using routine peptide or nucleotide chemistries and automated peptide or nucleotide synthesizers. Such routine methodologies are described for example in the following publications, the teachings of which are incorporated by reference herein: Hilvert, 1 Chem.-Biol. 201-3 (1994); Muir et al., 95 Proc. Natl. Acad. Sci. USA 6705-10 (1998); Wallace, 6 Cuff. Opin. Bioteclniol. 40310 (1995); Miranda et al., 96 Proc. Natl. Acad. Sci. USA 1181-86 (1999); Liu et al., 91 Proc. Natl. Acad. Sci. USA 6584-88 (1994). Suitable for use in the present disclosure are naturally-occurring amino acids and nucleotides; non-naturally occurring amino acids and nucleotides; modified or unusual amino acids; modified bases; amino acid sequences that contain post-translationally modified amino acids and/or modified linkages, cross-links and end caps, non-peptidyl bonds, etc.; and, further including without limitation, those moieties disclosed in the World intellectual Documentation. Standard St. 25 (1998) including Tables 1 through 6 in Appendix 2, herein incorporated by reference. Equivalents of the foregoing will be appreciated by the skilled artisan relying only on routine experimentation together with the knowledge of the art.

For example, the contemplated DNA constructs may be manufactured by the assembly of synthetic nucleotide sequences and/or joining DNA restriction fragments to produce a synthetic DNA molecule. The DNA molecules then are ligated into an expression vehicle, for example an expression plasmid, and transfected into an appropriate host cell, for example E. coli. The contemplated protein construct encoded by the DNA molecule then is expressed, purified, refolded, tested in vitro for certain attributes, e.g., binding activity with a receptor having binding affinity for the template TGF-beta superfamily member, and subsequently tested to assess whether the biosynthetic construct mimics other preferred attributes of the template superfamily member.

Alternatively, a library of synthetic DNA constructs can be prepared simultaneously for example, by the assembly of synthetic nucleotide sequences that differ in nucleotide composition. in a preselected region. For example, it is contemplated that during production of a construct based upon a specific TGF-beta superfamily member, the artisan can choose appropriate core or variable regions for such a superfamily member. Once the appropriate core or variable regions have been selected, the artisan then can produce synthetic DNA encoding these regions. For example, if a plurality of DNA molecules encoding different linker sequences are included into a ligation reaction containing DNA molecules encoding desired core domain and variable domain sequences, by judicious choice of appropriate restriction sites and reaction conditions, the artisan may produce a library of DNA constructs wherein each of the DNA constructs encode desired core domains or variable domains but connected by different linker sequences. The resulting DNAs then are ligated into a suitable expression vehicle, i.e., a plasmid useful in the preparation of a phage display library, transfected into a host cell, and the polypeptides encoded by the synthetic DNAs expressed to generate a pool of candidate-proteins. The pool of candidate proteins subsequently can be screened to identify specific proteins having the desired binding affinity and/or selectivity for a pre-selected receptor.

Screening can be performed by passing a solution comprising the candidate proteins through a chromatographic column containing surface immobilized receptor.

Then proteins with the desired binding specificity are eluted, for example by means of a salt gradient and/or a concentration gradient of the template TGF-beta superfamily member. Nucleotide sequences encoding such proteins subsequently can be isolated and characterized. Once the appropriate nucleotide sequences have been identified, the lead proteins subsequently can be produced, either by conventional recombinant DNA or peptide synthesis methodologies, in quantities sufficient to test whether the particular construct mimics the activity of the template TGF-beta superfamily member. It is contemplated that, which ever approach is adopted to produce DNA molecules encoding constructs of the disclosure, the tertiary structure of the preferred proteins can subsequently be modulated in order to optimize binding and/or biological activity by, for example, by a combination of nucleotide mutagenesis methodologies aided by the principles described herein and phage display methodologies. Accordingly, an artisan can produce and test simultaneously large numbers of such proteins.

The construction of DNAs encoding the biosynthetic constructs disclosed herein is performed using known techniques involving the use of various restriction enzymes which make sequence specific cuts in DNA to produce blunt ends or cohesive ends, DNA ligases, techniques enabling enzymatic addition of sticky ends to blunt-ended DNA, construction of synthetic DNAs by assembly of short or medium length oligonucleotides, cDNA synthesis techniques, polymerase chain reaction (PCR) techniques for amplifying appropriate nucleic acid sequences from libraries, and synthetic probes for isolating genes of members of the TGF-beta superfamily and their cognate receptors. Various promoter sequences from bacteria, mammals, or insects to name a few, and other regulatory DNA sequences used in achieving expression, and various types of host cells are also known and available. Conventional transfection techniques, and equally conventional techniques for cloning and subcloning DNA are useful in the practice of this disclosure and known to those skilled in the art. Various types of vectors may be used such as plasmids and viruses including animal viruses and bacteriophages. The vectors may exploit various marker genes that impart to a successfully transfected cell a detectable phenotypic property that can be used to identify which of a family of clones has successfully incorporated the recombinant DNA of the vector.

One method for obtaining DNA encoding the biosynthetic constructs disclosed herein is by assembly of synthetic oligonucleotides produced in a conventional, automated, oligonucleotide synthesizer followed by ligation with appropriate ligases.

For example, overlapping, complementary DNA fragments may be synthesized using phosphoramidite chemistry, with end segments left unphosphorylated to prevent polymerization during ligation. One end of the synthetic DNA is left with a “sticky end” corresponding to the site of action of a particular restriction endonuclease, and the other end is left with an end corresponding to the site of action of another restriction endonuclease. The complementary DNA fragments are ligated together to produce a synthetic DNA construct.

Alternatively nucleic acid strands encoding desired core or variable regions of a TGF-beta superfamily member can be isolated from libraries of nucleic acids, for example, by colony hybridization procedures such as those described in Sambrook et al., (1990) Molecular Cloning: A Laboratory Manual., 2d ed. (Cold Spring Harbor, N.Y.: Cold Spring Harbor Laboratory Press), and/or by PCR amplification methodologies, such as those disclosed in Innis et al. (1990) TM Protocols. “A guide to methods and applications,” Academic Press. The nucleic acids encoding the desired core or variable domains then are joined together to produce a synthetic DNA encoding the biosynthetic single-chain morphon construct of interest.

It is appreciated, however, that a library of DNA constructs encoding a plurality thereof can be produced simultaneously by standard recombinant DNA methodologies, such as the ones, described above, For example, the skilled artisan by the use of cassette mutagenesis or oligonucleotide directed mutagenesis can produce, for example, a series of DNA constructs each of which contain different DNA sequences within a predefined location, e.g., within a DNA cassette encoding a linker sequence. The resulting library of DNA constructs subsequently can be expressed, for example, in a phage or viral display library or a eukaryotic cell line (e.g., CHO cell line); and any protein constructs that binds to a specific receptor may be isolated by affinity purification, e.g., using a chromatographic column comprising surface immobilized receptor. Once molecules that bind the preselected receptor have been isolated, their binding and agonist properties can be modulated using empirical refinement techniques.

Methods of mutagenesis of proteins and nucleic acids are well known and well described in the art. See, e.g., Sambrook et al, supra. Useful methods include PCR (overlap extension, see, e.g., PCR Primer (Dieffenbach and Dveksler, eds., Cold Spring Harbor Press, Cold Spring Harbor, N.Y., 1995, pp. 603-611)); cassette mutagenesis and single-stranded mutagenesis following the method of Kunkel. It will be appreciated by the artisan that any suitable method of mutagenesis can be utilized and the mutagenesis method is not considered a material aspect of the disclosure. The nucleotide codons competent to encode amino acids, including arginine (Arg), glutamic acid (Glu) and aspartic acid (Asp) also are well known and described in the art. See, for example, Lehninger, Biochemistry (Worth publishers, N.Y., N.Y.). Standard codons encoding arginine, glutamic acid and aspartic acid are: Arg: CGU, CGC, CGA, CGG, AGA, AGG; Glu: GAA, GAG; and Asp: GAU, GAC. Mutant constructs of the disclosure can readily be constructed by aligning the nucleic acid sequences or domains to be switched, and identifying compatible splice sites and/or constructing suitable crossover sequences using PCR overlap extension.

Exemplary nucleic acid sequence of a TGF-beta superfamily member is shown as follows:

>gi|24475947|ref|NM_013611.2| Mus musculus nodal
(Nodal), mRNA
AAGCTTCACTCGGAGCAAGCCTTAGCCCGCTGTCTCAGCAGGGAGACTTC
CCGAGGTAGAGGGGCAAGGTGCGGGGCGGTTTAGACTCAGAGTCTGTATG
CACCCCTAACTCCCCCCCCCCCCCCCGCCACAATTTCTCTGTAGTCTTTC
TCAGCACATCACACCTCCCCTCAGCAGGGGCTCCCCTGCCCTGCCCCTCC
AGGGTGGTTATAAGTTCTTAACCTATAGGTTATAGGCCTCTCCGGAGGGA
CGGACGGAAAGGGGCGGGGCGGCCGCGGCTCAGATATAAGGGACCCTGGT
GTTGTTTGCGGGTCCAAACAGCCCACCATGAGTGCCCACAGCCTCCGCAT
CCTTCTTCTTCAAGCCTGTTGGGCTCTACTCCACCCGCGCGCCCCGACCG
CGGCCGCTTTGCCTCTGTGGACACGGGGGCAGCCCTCGTCACCGTCCCCT
CTGGCGTACATGTTGAGCCTCTACCGAGACCCGCTGCCTCGGGCGGACAT
CATCCGCAGCCTCCAGGCGCAAGATGTGGACGTGACCGGACAGAACTGGA
CTTTCACGTTTGACTTCTCCTTTTTGAGCCAAGAAGAGGATCTGGTATGG
GCGGACGTCCGGTTGCAGCTGCCGGGCCCCATGGACATACCCACTGAGGG
CCCACTCACCATTGACATTTTCCACCAGGCCAAGGGGCATCCAGAGCGGG
ACCCCGCTGACTGCCTGGAGCGCATTTGGATGGAGACGTTCACCGTCATT
CCTTCTCAGGTCACGTTTCCCTCAGGCAGCACAGTCCTGGAGGTGACCAA
CCCACTCTCCAAGTGGCTAAAGGACCCCAGCGCACTGGAAAAGCAGGTGT
CCAGTCGAGCAGAAAAGTGTTGGCATCAGCCCTACACCCCACCTGTACCT
GTCGCCAGCACCAATGTGCTCATGCTCTACTCCAACCGGCCTCAGGAGCA
GAGGCAGCTAGGGGGCGCCACTTTGCTTTGGGAAGCTGAGAGCTCCTGGC
GGGCCCAGGAGGGACAGCTGTCTGTAGAGAGGGGCGGATGGGGCAGAAGG
CAACGCCGACATCATTTGCCAGACAGAAGCCAACTGTGTAGGAGGGTCAA
GTTCCAGGTGGACTTCAACCTGATTGGCTGGGGCTCCTGGATCATCTACC
CCAAGCAGTACAATCCCTATCGCTGTGAGGGCGAGTGTCCTAACCCTGTG
CGGGAGGAGTTTCATCCTACCAACCATGCCTACATCCAGAGCCTGCTGAA
ACGATACCAACCCCACCGGGTTCCTTCCACGTGCTGTGCCCCCGTGAAGA
CCAAGCCACTGAGCATGCTTTATGTGGACAATGGCAGGGTCCTCCTGGAA
CACCACAAGGACATGATTGTGGAGGAGTGTGGGTGCCTCTGACAGAGCCA
GGGGGAGTGCTGAAATTGGCTTGCATTCCACAATGCTGATGAACTCCAAG
GAGACTCCATTGTGTCTATCCAGGGAGCAGAAACGTTAGAAGAGTTCTGC
CTGCTGGAGCTAAAGAGAAAAGCCCCGCCCCCTGTGCATACAGTGCTCTT
AGACCTGCCAAGCCAGAGAGAGGCTACCGTCGCATGGCAGGATGGGGAAG
CCTTGCAGGGGCTGGCTCGCTGGGCTCCCTGGAAATAGGGTTTATGAACT
GCTTGAAATTGTGTGCAAAGGCTGGGGTGTATATATATATATGTATATAT
ATATATATGTATTTATTTTTGATGCCATCTGTAACTTTCTTCTATTCTCA
AAAGTGGTCTGTGACCTGCTGTCCCTCCCTCAAGATTAGTATATATTTTA
TTAGATTATAAACGAGCCATTTGGTTCTCCCTGCCTCAAGCTGTGGTAGG
GAAGACCCACAACCTTCTGGCTGGCTGGCAGTGACATCCTGGCCTTGGTC
AGGGGCTCTCTGATCTCTAATGACTTCCCTAAAAAAGCCACTGTCCAGTT
CTCCAGGGCCAGTTGGTGCCTTTGACCAGAGAGGTGGGCACTTGTCCAAG
AGGGGACTGGCCATGGTGGACTTTAGAAGCCAGAGTCCTGAGATGTATGC
TTGGCAGACACAACCCAAGTCTATTAAAAGTCTGTGACAATTCAAAAAAA
AAAAAAAAAAAAAAAAAAAAAAAAAAAAAAAAAAAAAAAAAAAAAAAAAA
AAAAAAAAAA
Examples of nucleic acids encoding a core domain
of a TGF-beta superfamily protein include:

SEQ ID NO:32

CGACATCATTTGCCAGACAGAAGCCAACTGTGTAGGAGGGTCAAGTTCCA
GGTGGACTTCAACC

SEQ ID NO:33

TATCGCTGTGAGGGCGAGTGT

SEQ ID NO:34

TACCAACCCCACCGGGTTCCTTCCACGTGCTGTGCCCCCGTGAAGACCAA
G

SEQ ID NO:35

AAGGACATGATTGTGGAGGAGTGTGGGTGCCTC

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects of the disclosure, the subject nucleic acid is provided in an expression vector comprising a nucleotide sequence encoding a chimeric TGF-beta superfamily protein and operably linked to at least one regulatory sequence. Regulatory sequences are art-recognized and are selected to direct expression of the chimeric TGF-beta superfamily protein. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a chimeric protein of the disclosure. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant chimeric TGF-beta superfamily protein include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant SLC5A8 polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the 3-gal containing pBlueBac III).

In a preferred embodiment, a vector will be designed for production of a subject chimeric TGF-beta superfamily protein in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject chimeric protein in cells propagated in culture, e.g., to produce proteins, including fusion proteins or variant proteins, for purification.

This disclosure also pertains to a host cell transfected with a recombinant gene including a coding sequence for one or more of the subject chimeric TGF-beta superfamily proteins. The host cell may be any prokaryotic or eukaryotic cell. For example, a chimeric protein of the disclosure may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject chimeric TGF-beta superfamily proteins. For example, a host cell transfected with an expression vector encoding a subject chimeric protein can be cultured under appropriate conditions to allow expression of the subject chimeric protein to occur. The chimeric TGF-beta superfamily protein may be secreted and isolated from a mixture of cells and medium containing the chimeric polypeptide. Alternatively, the chimeric polypeptide may be retained cytoplasmically or in a membrane fraction and the cells harvested, lysed and the protein isolated. A cell culture includes host cells, media and other byproducts. Suitable media for cell culture are well known in the art. The chimeric polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the chimeric protein.

In a preferred embodiment, the chimeric TGF-beta superfamily protein is a fusion protein containing a domain which facilitates its purification. In certain embodiments, a fusion gene coding for a purification leader sequence, such as a poly-(His)/enterokinase cleavage site sequence at the N-terminus of the desired portion of the recombinant chimeric protein, can allow purification of the expressed fusion protein by affinity chromatography using a Ni²⁺ metal resin. The purification leader sequence can then be subsequently removed by treatment with enterokinase to provide the purified chimeric protein (e.g., see Hochuli et al., (1987) J. Chromatography 411:177; and Janknecht et al., PNAS USA 88:8972). Techniques for making fusion genes are well known are discussed above.

### 5. Assays for Functionality of Modified TGF-Beta Superfamily Proteins

In certain aspects, the present disclosure relates to assays testing biological activities (or effects) of a modified TGF-beta superfamily protein.

A modified protein of the disclosure may comprise an agonist of a TGF-beta superfamily protein or, alternatively, an antagonist of a TGF-beta superfamily protein.

In certain embodiments, a chimeric protein of the disclosure comprising an agonist of a TGF-beta superfamily protein comprises an antagonist of a different TGF-beta superfamily protein.

In certain embodiments, a chimeric protein of the disclosure comprises an agonist of the TGF-beta superfamily protein with which a common variable domain is shared. As is known in the art, such a chimeric protein may comprise an antagonist of another TGF-beta superfamily protein. For example, a chimeric protein of the disclosure comprising a variable domain from BMP-3 may comprise an agonist of BMP-3 and an antagonist of BMP-2.

In certain embodiments, a chimeric protein of the disclosure is an antagonist of the TGF-beta superfamily protein with which a common variable domain is shared.

As is known in the art, such a chimeric protein may comprise agonist of another TGF-beta superfamily protein. For example, a chimeric protein of the disclosure comprising a variable domain from BMP-3 may comprise antagonist of BMP-3 and an agonist of BMP-2.

The term “agonist,” as used herein, is meant to refer to a subject chimeric protein or compound that mimics or upregulates (e.g., potentiates or supplements) biological activity of a naturally-occurring TGF-beta superfamily protein. For example, an agonist can be a subject chimeric protein having at least one biological activity of naturally-occurring TGF-beta superfamily protein. Alternatively, an agonist of a subject chimeric protein can be a compound that mimics or upregulates at least one biological activity of the subject chimeric protein. An agonist of a TGF-beta protein can also be a protein or compound that upregulates expression of the TGF-beta protein or a gene regulated by the TGF-beta protein.

By “antagonist” herein is meant a subject chimeric protein or compound that downregulates (e.g., suppresses or inhibits) biological activity of a naturally-occurring TGF-beta superfamily protein. For example, an agonist can be a subject chimeric protein blocking or inhibiting at least one biological activity of naturally-occurring TGF-beta superfamily protein. Alternatively, an agonist of a subject chimeric protein can be a compound that downregulates at least one biological activity of the subject chimeric protein. An antagonist of a TGF-beta protein may also be a compound that downregulates expression of the TGF-beta protein or a gene regulated by the TGF-beta protein.

Irrespective of which protein expression, harvesting, and, folding methodologies are used, certain of the subject chimeric proteins can bind, preferentially to a pre-selected receptor and can now be identified using standard methodologies, i.e., ligand/receptor binding assays, well known, and thoroughly documented in the art. See, e.g., Legerski gl al. (1992) Bio h˜_Biophys. Res. Comm. 183: 672679; Frakar et al. (1978) Biochem. Bio12-hys. Res. Comm 80:849-857; Chio et el. (1990) Nature 343: 266-269; Dahlman et al. (1988) Biochem 27: 1813-1817; Strader et el. (1989) J. Biol. Chem. 264: 13572-13578; and DDowd et al. (1988) J. Biol. Chem. 263: 15985-15992.

Typically, in a ligand/receptor binding assay, the native or parent TGF-beta superfamily member of interest having a known, quantifiable affinity for a pre-selected receptor is labeled with a detectable moiety, for example, a radiolabel, a chromogenic label, or a fluorogenic label. Aliquots of purified receptor, receptor binding domain fragments, or cells expressing the receptor of interest on their surface are incubated with the labeled TGF-beta superfamily member in the presence of various concentrations of the unlabeled chimeric protein. The relative binding affinity of a candidate chimeric protein may be measured by quantitating the ability of the chimeric protein to inhibit the binding of the labeled TGF-beta superfamily member with the receptor. In performing the assay, fixed concentrations of the receptor and the TGF-beta superfamily member are incubated in the presence and absence of unlabeled chimeric protein. Sensitivity may be increased by preincubating the receptor with the chimeric protein before adding the labeled template TGF-beta superfamily member. After the labeled competitor has been added, sufficient time is allowed for adequate competitor binding, and then free and bound labeled TGF-beta superfamily members are separated from one another, and one or the other measured. Labels useful in the practice of the screening procedures include radioactive labels, chromogenic labels, spectroscopic labels such as those disclosed in Haughland (1994) “Handbook of Fluorescent and Research Chemicals,” 5 ed. by Molecular Probes, Inc., Eugene, Oreg., or conjugated enzymes having high turnover rates, i.e., horseradish peroxidase, alkaline phosphatase, or agalactosidase, used in combination with chemiluminescent or fluorogenic substrates. The biological activity, namely the agonist or antagonist properties of the resulting chimeric protein constructs can subsequently be characterized using conventional in vivo and in vitro assays that have been developed to measure the biological activity of any TGF-beta superfamily member. It is appreciated, however, the type of assay used preferably depends on the TGF-a superfamily member upon which the chimeric protein is based. For example, chimeric constructs based upon naturally occurring BMP-2 protein may be assayed using any of the biological assays that have been developed to date for measuring BMP-2 activity, described in more detail below.

The presence of dimers among the subject chimeric proteins can be detected visually either by standard SDS-PAGE in the absence of a reducing agent such as DTT or by HPLC (e.g., C18 reverse phase HPLC). Dimeric proteins of the present disclosure can have an apparent molecular weight in the range about 28-36 kDa, as compared to monomeric subunits, which may have an apparent molecular weight of about 14-18 kDa. The dimeric protein can readily be visualized on an electrophoresis gel by comparison to commercially available molecular weight standards. The dimeric protein also elutes from a C18 RP HPLC (45-50% acetonitrile: 0.1% TFA) at a time point different from that for its monomeric counterpart.

A second assay evaluates the presence of dimer (e.g., OP-1 dimers) by its ability to bind to hydroxyapatite. Optimally-folded dimer binds a hydroxyapatite column well in pH7, 10 mM phosphate, 6M urea, and 0.142M NaCl (dimer elutes at 0.25 M NaCl) as compared to monomer, which does not bind substantially at those concentrations (monomer elutes at 0.1M NaCl). A third assay evaluates the presence of dimer by the protein's resistant to trypsin or pepsin digestion. The folded dimeric species is substantially resistant to both enzymes, particularly trypsin, which cleaves only a small portion of the N-terminus of the mature protein, leaving a biologically active dimeric species only slightly smaller in size than the untreated dimer (each monomer in amino acids smaller after trypsin cleavage). By contrast, the monomers and misfolded dimers are substantially degraded. In the assay, the protein is subjected to an enzyme digest using standard conditions, e.g., digestion in a standard buffer such as 50 mM Tris buffer, pH 8, containing 4 M urea, 100 mM NaCl, 0.3% Tween-80 and 20 mM methylamine. Digestion is allowed to occur at 37° C. for on the order of 16 hours, and the product visualized by any suitable means, preferably SDS PAGE.

The biological activity of the subject chimeric proteins, for example the chimeric proteins having one or more variable domain from BMPs, can be assessed by any of a number of means as described in WO00/20607. For example, the protein's ability to induce endochondral bone formation can be evaluated using the well characterized rat subcutaneous bone assay. In the assay bone formation is measured by histology, as well as by alkaline phosphatase and/or osteoclacin production. In addition, osteogenic proteins having high specific bone forming activity, such as OP-1, BMP-2, BMR4, BMP-5 and BMP-6, also induce alkaline phosphatase activity in an in vitro rat osteoblast or osteosarcoma cell-based assay. Such assays are well described in the art. See, for example, Sabokdar of al. (1994) Bone and Mineral 27:57-67.; Knutsen et al. (1993) Biochem Biophvs Res. Commun 194:1352-1358; and Maliakal et al. (1994) Growth Factors 1:227-234).

By contrast, osteogenic proteins having low specific bone forming activity, such as CDMP-1 and CDMP-2, for example, do not induce alkaline phosphatase activity in the cell based osteoblast assay. The assay thus provides a ready method for evaluating biological activity of B1b9 mutants. For example, CDMP-1, CDMP-2 and CMDP-3 all are competent to induce bone formation, although with a lower specific activity than BMP-2, BW-4, BV-5, BMP-6 or OP-1. Conversely, BMP-2, BMP-4, BMP-5, BPylP-6 and OP-1 all can induce articular cartilage formation, albeit with a lower specific activity than CDMP-1, CDMP-2 or CDMP-3. Accordingly, a chimeric protein having one or more variable domain from CDMP, designed and described herein to be a chimeric protein competent to induce alkaline phosphatase activity in the cell-based assay, is expected to demonstrate a higher specific bone forming activity in the rat animal bioassay.

The chimeric protein's biological activity can also be readily evaluated by the protein's ability to inhibit epithelial cell growth. A useful, well characterized in vitro assay utilizes mink lung cells or melanoma cells. See WO00/20607. Other assays for other members of the TGF-beta superfamily are well described in the literature and can be performed without undue experimentation.

In certain aspects, the present disclosure provides methods and agents for control and maintain skeletal muscle mass in a host, preferably a human. Therefore, any chimeric protein of the disclosure that is expected to affect muscle-related function of a TGF-beta superfamily protein such as for example GDF-8 can be tested in whole cells or tissues, in vitro or in vivo, to confirm their ability to modulate skeletal muscle mass. GDF-8 (also known as myostatin) is a negative regulator of skeletal muscle growth. GDF-8 knockout mice have approximately twice the skeletal muscle mass of normal mice. The effects of increased muscle mass on bone modeling may be investigated, e.g., by examining bone mineral content (BMC) and bone mineral density (BMD) in the femora of female GDF-8 knockout mice. Dual-energy X-ray absorptiometry (DEXA) densitometry can be used to measure whole-femur BMC and BMD, and PQCT densitometry can be used to calculate BMC and BMD from cross-sections of tissues. Hamrick, Anat Rec. 2003 May; 272A(1):388-91. As is known in the art, a chimeric protein of the disclosure may be introduced into the GDF-8 knockout mice, and similar assays can be used to determine the effect of the chimeric protein on skeletal muscle mass and bone density.

The dystrophic phenotype in the mdx mouse model of Duchenne muscular dystrophy (DMD) may also be employed to test the biological activity of a chimeric protein of the disclosure. It was reported that blockade of endogenous myostatin by using intraperitoneal injections of blocking antibodies for three months resulted in an increase in body weight, muscle mass, muscle size and absolute muscle strength in mdx mouse muscle along with a significant decrease in muscle degeneration and concentrations of serum creatine kinase. Bogdanovich et al., Nature. 2002 Nov. 28; 420(6914):418-21. Similar study may be employed to determine whether a chimeric protein of the disclosure potentiates or inhibits the endogenous GDF-8 activity.

In certain aspects, the present disclosure provides methods and agents for modulating neurogenesis. For example, GDF-11 is known to inhibit olfactory epithelium neurogenesis in vitro by inducing p27(Kip1) and reversible cell cycle arrest in progenitors. Wu et al. Neuron. 2003 Jan. 23; 37(2):197-207. The effect of a chimeric protein of the disclosure on neurogenesis can be similarly tested. Further, the effect of a chimeric protein of the disclosure on GDF-11's effect on neurogenesis can also be tested using similar assays as described in Wu et al. Id.

In certain aspects, the present disclosure provides methods and agents for stimulating bone formation and increasing bone mass. Therefore, any chimeric protein of the disclosure that is expected to affect bone-related function of a TGF-beta superfamily protein such as for example BMP-2, BMP-3, GDF-10, BMP-4, BMP-7, or BMP-8, can be tested in whole cells or tissues, in vitro or in vivo, to confirm their ability to modulate bone or cartilage growth. Various methods known in the art can be utilized for this purpose.

For example, BMP-3 inhibits BMP2-mediated induction of Msx2 and blocks BMP2-mediated differentiation of osteoprogenitor cells into osteoblasts. Thus, the effect of a subject chimer protein, preferably one comprising a variable domain from a BMP-2 or BMP-3, on bone or cartilage growth can be determined by their effect on the osteogenic activity of BMP-2, for example, by measuring induction of Msx2 or differentiation of osteoprogenitor cells into osteoblasts in cell based assays (see, e.g., Daluiski et al., Nat. Genet. 2001, 27(1):84-8; Hino et al., Front Biosci. 2004, 9:1520-9). Similarly, a subject chimeric protein, preferably one comprising a variable domain from a BMP-2 or BMP-3, may be tested for its osteogenic or anti-osteogenic activity or its agonistic or antagonistic effect on BMP-2-mediated osteogenesis.

Another example of cell-based assays includes analyzing the osteogenic or anti-osteogenic activity of a subject chimeric and test compounds in mesenchymal progenitor and osteoblastic cells. To illustrate, recombinant adenoviruses expressing a subject chimeric protein were constructed to infect pluripotent mesenchyimal progenitor C3H10T1/2 cells, preosteoblastic C2C12 cells, and osteoblastic TE-85 cells. Osteogenic activity is then determined by measuring the induction of alkaline phosphatase, osteocalcin, and matrix mineralization (see, e.g., Cheng et al., J bone Joint Surg Am. 2003, 85-A(8):1544-52).

Further, the present disclosure contemplates in vivo assays to measure bone or cartilage growth. For example, Namkung-Matthai et al., Bone, 28:80-86 (2001) discloses a rat osteoporotic model in which bone repair during the early period after fracture is studied. Kubo et al., Steroid Biochemistry & Molecular Biology, 68:197-202 (1999) also discloses a rat osteoporotic model in which bone repair during the late period after fracture is studied. These references are incorporated by reference herein in their entirety for their disclosure of rat model for study on osteoporotic bone fracture. In certain aspects, the present disclosure makes use of fracture healing assays that are known in the art. These assays include fracture technique, histological analysis, and biomechanical analysis, which are described in, for example, U.S. Pat. No. 6,521,750, which is incorporated by reference in its entirety for its disclosure of experimental protocols for causing as well as measuring the extent of fractures, and the repair process.

It is understood that the screening assays of the present disclosure apply to not only the subject chimeric proteins and variants thereof, but also any test compounds including agonists and antagonist of the chimeric proteins or their variants themselves. Further, these screening assays are useful for drug target verification and quality control purposes.

In other aspects, the present disclosure relates to the use of the subject chimeric TGF-beta superfamily proteins to identify compounds which can modulate activities of the chimeric proteins. Compounds identified through this screening can be tested in tissues (e.g., bone and/or cartilage) or cells (e.g., muscle cells) to assess their ability to modulate the test tissues or cells (e.g., bone/cartilage growth or muscle cell growth) in vitro. Optionally, these compounds can further be tested in animal models to assess their ability to modulate, e.g., bone/cartilage growth or muscle control and maintenance in vivo.

A variety of assay formats will suffice and, in light of the present disclosure, those not expressly described herein will nevertheless be comprehended by one of ordinary skill in the art. As described herein, the test compounds (agents) of the disclosure may be created by any combinatorial chemical method. Alternatively, the subject compounds may be naturally occurring biomolecules synthesized in vivo or in vitro. Compounds (agents) to be tested for their ability to act as modulators of bone or cartilage growth can be produced, for example, by bacteria, yeast, plants or other organisms (e.g., natural products), produced chemically (e.g., small molecules, including peptidomimetics), or produced recombinantly. Test compounds contemplated by the present disclosure include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase, photoactivatible crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a chimeric TGF-beta superfamily protein and its binding protein (e.g., the chimeric protein itself or a TGF-beta receptor protein or fragments thereof).

Merely to illustrate, in an exemplary screening assay of the present disclosure, the compound of interest is contacted with an isolated and purified chimeric protein which is ordinarily capable of binding to a TGF-beta receptor protein or fragments thereof, as appropriate for the intention of the assay. To the mixture comprising a subject chimeric protein and a TGF-beta receptor protein is then added a composition containing a test compound. Detection and quantification of the chimeric protein receptor complexes provides a means for determining the compound's efficacy at inhibiting (or potentiating) complex formation between the chimeric TGF-beta superfamily protein and its binding protein, e.g., the TGF-beta receptor or fragments thereof. The efficacy of the compound can be assessed by generating dose response curves from data obtained using various concentrations of the test compound. Moreover, a control assay can also be performed to provide a baseline for comparison. For example, in a control assay, an isolated and purified chimeric TGF-beta superfamily protein is added to a composition (cell-free or cell-based) containing a TGF-beta receptor protein or fragment thereof, and the formation of the chimeric protein-receptor complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system. Alternatively, cells expressing a TGF-beta receptor protein or fragments thereof on their surfaces can be used in certain assays.

Complex formation between a subject chimeric TGF-beta superfamily protein and its binding protein may be detected by a variety of techniques. For instance, modulation of the formation of complexes can be quantitated using, for example, detectably labeled proteins such as radiolabelled (e.g., ³²P, ³⁵S, ¹⁴C or ³H), fluorescently labeled (e.g., FITC), or enzymatically labeled chimeric protein or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a chimeric TGF-beta superfamily protein and its binding protein (e.g., a TGF-beta receptor protein or fragments thereof). Further, other modes of detection such as those based on optical waveguides (PCT Publication WO 96/26432 and U.S. Pat. No. 5,677,196), surface plasmon resonance (SPR), surface charge sensors, and surface force sensors are compatible with many embodiments of the disclosure.

Moreover, the present disclosure contemplates the use of an interaction trap assay, also known as the “two hybrid assay,” for identifying agents that disrupt or potentiate interaction between a chimeric TGF-beta superfamily protein and its binding protein (e.g., a TGF-beta receptor protein or fragments thereof). See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696).

### 6. Methods of Administration

In certain embodiments, compositions (e.g., those comprising the subject modified chimeric or non-chimeric proteins) of the present disclosure can be used for treating or preventing a disease or condition that is associated with abnormal or aberrant activity of a TGF-beta superfamily member protein or gene. These diseases, disorders, or conditions are generally referred to herein as “TGF-beta-associated conditions,” or more specifically based on the specific TGF-beta superfamily member involved, “Nodal-associated conditions,” “BMP-2-associated conditions,” “BMP-3-associated conditions,” “GDF-8-associated conditions,” “GDF-11-associated conditions,” “BMP-10-associated conditions,” etc. In certain embodiments, the present disclosure provides methods of treating or preventing an individual in need thereof through administering to the individual a therapeutically effective amount of a chimeric TGF-beta superfamily protein as described above. These methods are particularly aimed at therapeutic and prophylactic treatments of animals, and more particularly, humans.

Examples of conditions associated with specific TGF-beta superfamily members are provided as follows.

a. Bone-Related Conditions

Increased BMP (e.g., BMP-2 or BMP-4) activity can be explored for the treatment of a variety of disease conditions in which BMP activity is needed. Increased BMP (e.g., BMP-2 or BMP-4) activity may also be achieved by antagonizing activity of certain proteins such as for example BMP-3. For example, osteoporosis is a bone disorder characterized by the loss of bone mass, which leads to fragility and porosity of the bone of man. As a result, patients suffering from osteoporosis have an increased fracture risk of the bones. Postmenopausal women are particularly at risk for osteoporosis as a result of reduced levels of estrogen production. When administered at low levels, estrogens have a beneficial effect on the loss of bone.

BMP-7 (also known as OP-1) induces all markers of osteoblast differentiation in pluripotential and mesenchymal stem cells. BMP-7 and BMP-8 (OP-2) are reported to have high sequence similarity to other BMPs, such as for example BMP-2 or BMP-4. Based on a high degree of amino acid sequence homology, BMP-5, BMP-6, and BMP-7 are recognized as a subfamily of the BMPs. Complete deletion of BMP-5 coding sequences is compatible with viability. Mutations at the “short ear” locus are associated with a specific spectrum of morphologic alterations in the ear and many internal skeletal structures. Thus, modulating activities of BMP-5, BMP-6, BMP-7 and/or BMP-8 as well as other BMPs can provide new ways of treating bond-related conditions.

GDF5 (also known as CDMP1) is predominantly expressed at sites of skeletal morphogenesis. Transgenic mice expressing recombinant CDMP1 died before or just after birth and exhibited chondrodysplasia with expanded primordial cartilage, which consisted of an enlarged hypertrophic zone and a reduced proliferating chondrocyte zone, not only in the limbs but also in the axial skeleton. Tsumaki et al., J. Cell Biol. 144: 161-173, 1999.

In certain embodiments, compositions (e.g., the subject chimeric proteins) of the present disclosure can be used for inducing bone and/or cartilage formation, preventing bone loss, increasing bone mineralization or preventing the demineralization of bone. For example, the subject chimeric proteins and compounds identified in the present disclosure have application in treating osteoporosis and the healing of bone fractures and cartilage defects in humans and other animals. Subject chimeric proteins such as for example an mNodal-BMP-2 (if an agonist of naturally-occurring BMP-2) or an mNodal-BMP-3 (if an antagonist of naturally-occurring BMP-3) may be useful in patients that are diagnosed with subclinical low bone density, as a protective measure against the development of osteoporosis.

In a certain embodiment, the present disclosure provides methods of treating or preventing an individual suffering from a disease (disorder or condition) that is related to bone/cartilage defects through administering to the individual a therapeutically effective amount of a subject chimeric protein as described above. These methods are particularly aimed at therapeutic and prophylactic treatments of animals, and more particularly, humans.

In certain embodiment, methods and compositions of the present disclosure may find medical utility in the healing of bone fractures and cartilage defects in humans and other animals. The subject methods and compositions may also have prophylactic use in closed as well as open fracture reduction and also in the improved fixation of artificial joints. De novo bone formation induced by an osteogenic agent contributes to the repair of congenital, trauma-induced, or oncologic resection induced craniofacial defects, and also is useful in cosmetic plastic surgery. Further, methods and compositions of the disclosure may be used in the treatment of periodontal disease, and in other tooth repair processes. In certain cases, a subject chimeric protein may provide an environment to attract bone-forming cells, stimulate growth of bone-forming cells or induce differentiation of progenitors of bone-forming cells. Chimeric proteins of the disclosure may also be useful in the treatment of osteoporosis. Further, the subject chimeric proteins may be used in cartilage defect repair and prevention/reversal of osteoarthritis.

In certain embodiment, methods and compositions of the disclosure may also be used in wound healing and related tissue repair. The types of wounds include, but are not limited to, burns, incisions and ulcers. See e.g., PCT Publication No. WO84/01106. In certain embodiment, the disclosure provides a therapeutic method and composition for repairing fractures and other conditions related to cartilage and/or bone defects or periodontal diseases.

In certain specific embodiments, methods and compositions (e.g., the subject chimeric proteins) of the disclosure can be applied to conditions causing bone loss such as osteoporosis, hyperparathyroidism, Cushing's disease, thyrotoxicosis, chronic diarrheal state or malabsorption, renal tubular acidosis, or anorexia nervosa. Many people know that being female, having a low body weight, and leading a sedentary lifestyle are risk factors for osteoporosis (loss of bone mineral density, leading to fracture risk). However, osteoporosis can also result from the long-term use of certain medications. Osteoporosis resulting from drugs or another medical condition is known as secondary osteoporosis. In a condition known as Cushing's disease, the excess amount of cortisol produced by the body results in osteoporosis and fractures. The most common medications associated with secondary osteoporosis are the corticosteroids, a class of drugs that act like cortisol, a hormone produced naturally by the adrenal glands. Although adequate levels of thyroid hormones (which are produced by the thyroid gland) are needed for the development of the skeleton, excess thyroid hormone can decrease bone mass over time. Antacids that contain aluminum can lead to bone loss when taken in high doses by people with kidney problems, particularly those undergoing dialysis. Other medications that can cause secondary osteoporosis include phenyloin (Dilantin) and barbiturates that are used to prevent seizures; methotrexate (Rheumatrex, Immunex, Folex PFS), a drug for some forms of arthritis, cancer, and immune disorders; cyclosporine (Sandimmune, Neoral), a drug used to treat some autoimmune diseases and to suppress the immune system in organ transplant patients; luteinizing hormone-releasing hormone agonists (Lupron, Zoladex), used to treat prostate cancer and endometriosis; heparin (Calciparine, Liquaemin), an anticlotting medication; and cholestyramine (Questran) and colestipol (Colestid), used to treat high cholesterol. Gum disease causes bone loss because these harmful bacteria in our mouths force our bodies to defend against them. The bacteria produce toxins and enzymes under the gum-line, causing a chronic infection.

In certain embodiments, the present disclosure provides methods and therapeutic agents, for example, antagonists of BMP-2 or agonist of BMP-3, for treating diseases or disorders associated with abnormal or unwanted bone growth. For example, patients having the disease known as Fibrodysplasia Ossificans Progressiva (FOP) grow an abnormal “second skeleton” that prevents any movement. Overexpression of BMP-4 was noted in FOP patients. Additionally, abnormal bone growth can occur after hip replacement surgery and thus ruin the surgical outcome. This is a more common example of pathological bone growth and a situation in which antagonists of BMP-2 or 4, or agonist of BMP-3 may be therapeutically useful. Antagonists of BMP-2 or 4, or agonists of BMP-3 may also be useful for treating other forms of abnormal bone growth, such as the pathological growth of bone following trauma, burns or spinal cord injury. In addition, antagonists of BMP-2 or 4, or agonists of BMP-3 may be useful for treating or preventing the undesirable actions of BMPs associated with the abnormal bone growth seen in connection with metastatic prostate cancer or osteosarcoma. Examples of these antagonists of BMP-2 or 4, or agonists of BMP-3 include, but are not limited to, a first subject chimeric protein that is an antagonist of BMP-2 or 4, a second subject chimeric protein that is agonist of BMP-3, a compound that is agonist of the first chimeric protein, or a compound that is an agonist of the second chimeric protein.

In certain embodiments of the subject methods, one or more chimeric proteins can be administered, together (simultaneously) or at different times (sequentially or overlapping). In addition, a subject chimeric protein can be administered with another type of osteogenic, cartilage-inducing or bone-inducing factor. The two types of compounds may be administered simultaneously or at different times. It is expected that the chimeric proteins of the disclosure may act in concert with or perhaps synergistically with other osteogenic, cartilage-inducing or bone-inducing factors. A variety of osteogenic, cartilage-inducing and bone-inducing factors have been described, particularly bisphosphonates. See e.g., European Patent Application Nos. 148,155 and 169,016. For example, other factors that can be combined with the subject chimeric proteins include various growth factors such as epidermal growth factor (EGF), platelet derived growth factor (PDGF), transforming growth factors (TGF-α and TGF-β, and insulin-like growth factor (IGF).

b. Nodal-Associated Conditions

As Nodal is essential for mesoderm formation and subsequent organization of axial structures in early mouse development, exemplary nodal-associated conditions may include developmental processes such as the correct formation of various structures or in one or more post-developmental capacities including sexual development, pituitary hormone production, and creation of bone and cartilage. Nodal-associated conditions also include disorders of cell growth and differentiation such as inflammation, allergy, autoimmune diseases, infectious diseases, and tumors.

c. Skeletal Muscle-related Conditions

Exemplary GDF-8-associated conditions include, but are not limited to, neuromuscular disorders (e.g., muscular dystrophy and muscle atrophy), congestive obstructive pulmonary disease, muscle wasting syndrome, sarcopenia, cachexia, adipose tissue disorders (e.g., obesity), type 2 diabetes, and bone degenerative disease (e.g., osteoporosis).

Exemplary GDF-11-associated conditions include, but are not limited to, musculodegenerative and neuromuscular disorders, tissue repair (e.g., wound healing), neurodegenerative diseases (e.g., amyotrophic lateral sclerosis), immunologic disorders (e.g., disorders related to abnormal proliferation or function of lymphocytes), and obesity or disorders related to abnormal proliferation of adipocytes.

In certain embodiments, compositions (e.g., the subject chimeric proteins) of the disclosure are used as part of a treatment for a muscular dystrophy. The term “muscular dystrophy” refers to a group of degenerative muscle diseases characterized by gradual weakening and deterioration of skeletal muscles and sometimes the heart and respiratory muscles. Muscular dystrophies are genetic disorders characterized by progressive muscle wasting and weakness that begin with microscopic changes in the muscle. As muscles degenerate over time, the person's muscle strength declines. Exemplary muscular dystrophies that can be treated with a regimen including an appropriate modified TGF-beta superfamily member include: Duchenne Muscular Dystrophy (DMD), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (also known as Landouzy-Dejerine), Myotonic Dystrophy (MMD) (also known as Steinert's Disease), Oculopharyngeal Muscular Dystrophy (OPMD), Distal Muscular Dystrophy (DD), Congenital Muscular Dystrophy (CMD).

Recent researches demonstrate that blocking or eliminating GDF-8 function in vivo can effectively treat at least certain symptoms in DMD and BMD patients (Bogdanovich et al., supra). Thus, the subject chimeric proteins or compounds that are antagonists of naturally-occurring GDF-8 constitute an alternative means of blocking the functions of GDF-8 (and/or GDF-11) in vivo in DMD and BMD patients.

Similarly, the subject chimeric proteins provide an effective means to increase muscle mass in other disease conditions that are in need of muscle growth. For example, Gonzalez-Cadavid et al. (supra) reported that that GDF-8 expression correlates inversely with fat-free mass in humans and that increased expression of the GDF-8 gene is associated with weight loss in men with AIDS wasting syndrome. By inhibiting the function of GDF-8 in AIDS patients, at least certain symptoms of AIDS may be alleviated, if not completely eliminated, thus significantly improving quality of life in AIDS patients.

Since loss of GDF-8 function is also associated with fat loss without diminution of nutrient intake, the chimeric proteins may further be used as a therapeutic agent for slowing or preventing the development of obesity and type II diabetes.

The cancer anorexia-cachexia syndrome is among the most debilitating and life-threatening aspects of cancer. Progressive weight loss in cancer anorexia-cachexia syndrome is a common feature of many types of cancer and is responsible not only for a poor quality of life and poor response to chemotherapy, but also a shorter survival time than is found in patients with comparable tumors without weight loss. Associated with anorexia, fat and muscle tissue wasting, psychological distress, and a lower quality of life, cachexia arises from a complex interaction between the cancer and the host. It is one of the most common causes of death among cancer patients and is present in 80% at death. It is a complex example of metabolic chaos effecting protein, carbohydrate, and fat metabolism. Tumors produce both direct and indirect abnormalities, resulting in anorexia and weight loss. Currently, there is no treatment to control or reverse the process. Cancer anorexia-cachexia syndrome affects cytokine production, release of lipid-mobilizing and proteolysis-inducing factors, and alterations in intermediary metabolism. Although anorexia is common, a decreased food intake alone is unable to account for the changes in body composition seen in cancer patients, and increasing nutrient intake is unable to reverse the wasting syndrome. Cachexia should be suspected in patients with cancer if an involuntary weight loss of greater than five percent of premorbid weight occurs within a six-month period.

Since systemic overexpression of GDF-8 in adult mice was found to induce profound muscle and fat loss analogous to that seen in human cachexia syndromes, subject pharmaceutical compositions can be beneficially used to prevent, treat, or alleviate the symptoms of the cachexia syndrome, where muscle growth is desired.

TGF-beta 1 expression as measured by mRNA was greater in Duchenne Muscular Dystrophy and Becker Muscular Dystrophy patients than in controls. TGF-beta 1 has been implicated also in the pathogenesis of adult respiratory distress syndrome, and the kidney seems to be particularly sensitive to TGF-beta 1-induced fibrogenesis. TGF-beta 1 appears to play a role in the development of renal hypertrophy and accumulation of extracellular matrix in diabetes. It is known to have powerful fibrogenic actions. In both humans and animal models, TGF-beta 1 mRNA and protein levels are significantly increased in the glomeruli and tubulointerstitium in diabetes.

d. Other Conditions

GDF-1 knockout mice exhibited a spectrum of defects related to left-right axis formation, including visceral situs inversus, right pulmonary isomerism, and a range of cardiac anomalies.

GDF-7, a BMP family member expressed selectively by roof plate cells, in the generation of neuronal cell types in the dorsal spinal cord. GDF-7 can promote the differentiation in vitro of two dorsal sensory interneuron classes, DIA and DIB neurons. In Gdf7-null mutant embryos, the generation of D1A neurons is eliminated but D1B neurons and other identified dorsal interneurons are unaffected. These findings show that GDF-7 is an inductive signal from the roof plate required for the specification of neuronal identity in the dorsal spinal cord and that GDF-7 and other BMP family members expressed by the roof plate have non-redundant functions in vivo. Lee et al. Genes Dev. 1998 Nov. 1; 12(21):3394-407.

Of the 3 TGF-betas, TGF-beta 1 is most frequently upregulated in tumor cells and is the focus of most studies on the role of TGF-beta 1 in tumorigenesis. Scleroderma is a chronic systemic disease that leads to fibrosis of the skin and other affected organs. TGF-beta 1 has been implicated in the pathogenesis of scleroderma.

A decreased level of GDF9 signal was observed in developing polycystic ovary oocytes, compared with normal; GDF9B (also known as BMP-15) is essential for female fertility and that natural mutations in an ovary-derived factor can cause both increased ovulation rate and infertility phenotypes in a dosage-sensitive manner. Thus, exemplary GDF-9- or BMP-15-associated conditions include, but are not limited to, disorders associated with female fertility.

In certain embodiments, the subject chimeric proteins can be used to form pharmaceutical compositions that can be beneficially used to prevent, treat, or alleviate symptoms of a host of diseases involving neurodegeneration. While not wishing to be bound by any particular theory, a subject chimeric protein may antagonize the inhibitory feedback mechanism mediated through the wild-type ALK7 receptor, thus allowing new neuronal growth and differentiation. The subject chimeric proteins as pharmaceutical compositions can be beneficially used to prevent, treat, or alleviate symptoms of diseases with neurodegeneration, including Alzheimer's Disease (AD), Parkinson's Disease (PD), Amyotrophic Lateral Sclerosis (ALS), and Huntington's disease (HD).

AD is a chronic, incurable, and unstoppable central nervous system (CNS) disorder that occurs gradually, resulting in memory loss, unusual behavior, personality changes, and a decline in thinking abilities. PD is a chronic, incurable, and unstoppable CNS disorder that occurs gradually and results in uncontrolled body movements, rigidity, tremor, and gait difficulties. These motor system problems are related to the death of brain cells in an area of the brain that produces dopamine, a chemical that helps control muscle activity.

ALS, also called Lou Gehrig's disease (motor neuron disease) is a chronic, incurable, and unstoppable CNS disorder that attacks the motor neurons, components of the CNS that connect the brain to the skeletal muscles. In ALS, the motor neurons deteriorate and eventually die, and though a person's brain normally remains fully functioning and alert, the command to move never reaches the muscles.

HD is another neurodegenerative disease resulting from genetically programmed degeneration of neurons in certain areas of the brain. This degeneration causes uncontrolled movements, loss of intellectual faculties, and emotional disturbance.

Tay-Sachs disease and Sandhoff disease are glycolipid storage diseases caused by the lack of lysosomal β-hexosaminidase (Gravel et al., in The Metabolic Basis of Inherited Disease, eds. Scriver et al., McGraw-Hill, New York, pp. 2839-2879, 1995). In both disorders, GM2 ganglioside and related glycolipidssubstrates for r-hexosaminidase accumulate in the nervous system and trigger acute neurodegeneration. In the most severe forms, the onset of symptoms begins in early infancy. A precipitous neurodegenerative course then ensues, with affected infants exhibiting motor dysfunction, seizure, visual loss, and deafness.

It is well-known that apoptosis plays a role in AIDS pathogenesis in the immune system. However, HIV-1 also induces neurological disease. Shi et al. (J. Clin. Invest. 98: 1979-1990, 1996) examined apoptosis induced by HIV-1 infection of the central nervous system (CNS) in an in vitro model and in brain tissue from AIDS patients, and found that HIV-1 infection of primary brain cultures induced apoptosis in neurons and astrocytes in vitro. Apoptosis of neurons and astrocytes was also detected in brain tissue from 10/11 AIDS patients, including 5/5 patients with HIV-1 dementia and 4/5 nondemented patients.

Neuronal loss is a also a salient feature of prion diseases, such as Creutzfeldt-Jakob disease in human, BSE in cattle (mad cow disease), Scrapie Disease in sheep and goats, and feline spongiform encephalopathy (FSE) in cats.

The subject chimeric proteins are also useful to prevent, treat, and alleviate symptoms of various PNS disorders, such as the ones described below. The PNS is composed of the nerves that lead to or branch off from the CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used. Peripheral neuropathy is a widespread disorder, and there are many underlying causes. Some of these causes are common, such as diabetes, and others are extremely rare, such as acrylamide poisoning and certain inherited disorders. The most common worldwide cause of peripheral neuropathy is leprosy. Leprosy is caused by the bacterium Mycobacterium leprae, which attacks the peripheral nerves of affected people.

Another of the better known peripheral neuropathies is Guillain-Barré syndrome, which arises from complications associated with viral illnesses, such as cytomegalovirus, Epstein-Barr virus, and human immunodeficiency virus (HIV), or bacterial infection, including Campylobacter jejuni and Lyme disease. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the peripheral nervous system (PNS) and the central nervous system (CNS), as well as other body tissues.

Other PNS diseases treatable with the subject chimeric proteins include: Brachial Plexus Neuropathies (diseases of the cervical and first thoracic roots, nerve trunks, cords, and peripheral nerve components of the brachial plexus. Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp 1351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy (see Adams et al., Principles of Neurology, 6th ed, p 1325); mononeuropathies (disease or trauma involving a single peripheral nerve in isolation, or out of proportion to evidence of diffuse peripheral nerve dysfunction). Mononeuropathy multiplex refers to a condition characterized by multiple isolated nerve injuries. Mononeuropathies may result from a wide variety of causes, including ischemia; traumatic injury; compression; connective tissue diseases; cumulative trauma disorders; and other conditions); Neuralgia (intense or aching pain that occurs along the course or distribution of a peripheral or cranial nerve); Peripheral Nervous System Neoplasms (neoplasms which arise from peripheral nerve tissue. This includes neurofibromas; Schwalnomas; granular cell tumors; and malignant peripheral nerve sheath tumors. See DeVita Jr et al., Cancer: Principles and Practice of Oncology, 5th ed, pp 1750-1); Nerve Compression Syndromes (mechanical compression of nerves or nerve roots from internal or external causes. These may result in a conduction block to nerve impulses, due to, for example, myelin sheath dysfunction, or axonal loss. The nerve and nerve sheath injuries may be caused by ischemia; inflammation; or a direct mechanical effect); Neuritis (a general term indicating inflammation of a peripheral or cranial nerve. Clinical manifestation may include pain; paresthesias; paresis; or hyperthesia); Polyneuropathies (diseases of multiple peripheral nerves. The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

In certain embodiments, compositions of the present disclosure can be used for treating or preventing a disease or condition that is associated with abnormal activity of BMP10. These diseases, disorders, or conditions are generally referred to herein as “BMP10-associated conditions.”

In one embodiment, the present invention provides methods for treating or preventing heart disorders in a subject. BMP-10 is associated with proliferation and growth of cardiomyocytes, and certain conditions may be treated by administering a BMP-10 agonist so as to stimulate cardiomyocyte growth. Such conditions include essentially any condition associated with death of cardiomyocytes, such ischemic damage associated with, e.g., myocardial infarction. Physical or inflammatory damage to heart muscle may also be treated with a BMP-10 agonist.

As described herein, a “cardiomyocyte” is a cell of the cardiac muscle that is striated like skeletal muscle, having microscopically visible myofilaments arranged in parallel with the sarcomere. Cardiac muscle can generate its own excitatory impulses from the sino-atrial node, which acts like a biological pacemaker. In this manner, the contracting signal for cardiac muscles originates in the heart itself. However, the autonomic nervous system can exert control over how fast the signals form and propagate through the heart, which regulates the rate of myocardial contraction.

In certain embodiments, a modified protein of the disclosure may be locally administered to promote regeneration of cardiac tissue damaged post myocardial infarction. Preferably, a modified protein comprising core regions from a Nodal protein and variable regions from a BMP-10 protein (e.g., the chimer protein comprising an amino acid sequence of SEQ ID NO:14) may be locally administered to promote regeneration of cardiac tissue damaged post myocardial infarction.

### 7. Pharmaceutical Compositions

In certain embodiments, compounds (e.g., modified chimeric or non-chimeric TGF-beta polypeptides) of the present disclosure are formulated with a pharmaceutically acceptable carrier. For example, a TGF-beta chimeric polypeptide can be administered alone or as a component of a pharmaceutical formulation (therapeutic composition). The subject compounds may be formulated for administration in any convenient way for use in human or veterinary medicine.

In certain embodiments, the therapeutic method of the disclosure includes administering the composition topically, systemically, or locally, e.g., as an implant or device. When administered, the therapeutic composition for use in this disclosure is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than the modified TGF-beta polypeptides which may also optionally be included in the composition as described above, may alternatively or additionally, be administered simultaneously or sequentially with the modified TGF-beta polypeptides in the methods of the disclosure. Preferably for bone or cartilage formation, the composition would include a matrix capable of delivering the modified TGF-beta polypeptides or other therapeutic compounds to the site of bone or cartilage damage, providing a structure for the developing bone and cartilage and optimally capable of being resorbed into the body. For example, the matrix may provide slow release of the modified TGF-beta polypeptides. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalciumphosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

In certain embodiments, methods of the disclosure can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, or any of the following: (1) fillers or extenders, such as starches, lactose, sucrose, glucose, mannitol, or silicic acid; (2) binders, such as, for example, carboxymethylcellulose, alginates, gelatin, polyvinyl pyrrolidone, sucrose, or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the disclosure (e.g., a TGF-beta chimeric polypeptide), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, pharmaceutical compositions suitable for parenteral administration may comprise one or more modified TGF-beta polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain embodiments, pharmaceutical compositions suitable for local administration may comprise one or more modified TGF-beta polypeptides in combination with a pharmaceutically or physiologically acceptable carrier. For example, a chimeric protein of the disclosure comprising core regions from a Nodal protein and variable regions from a BMP-10 protein may be formulated suitable for local administration to promote regeneration of cardiac tissue damaged post myocardial infarction

The compositions of the disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., modified chimeric or non-chimeric TGF-beta polypeptides). The various factors include, but are not limited to, amount of bone weight desired to be formed, the site of bone damage, the condition of the damaged bone, the size of a wound, type of damaged tissue, the patient's age, sex, and diet, the severity of any infection, time of administration, and other clinical factors. Optionally, the dosage may vary with the type of matrix used in the reconstitution and the types of compounds in the composition. The addition of other known growth factors to the final composition, may also effect the dosage. Progress can be monitored by periodic assessment of bone growth or repair, for example, X-rays, histomorphometric determinations, and tetracycline labeling.

### INCORPORATION BY REFERENCE

All publications including patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

1. A chimeric TGF-beta superfamily protein comprising a core domain from a first TGF-beta superfamily member and a variable domain from a second TGF-beta superfamily member, wherein said chimeric protein binds to at least one of TGF-beta type 1 receptor and TGF-beta type 2 receptor.

2. The chimeric protein of claim 1, wherein said chimer protein forms a homodimer.

3. The chimeric protein of claim 1, wherein said chimeric protein is an agonist or antagonist of the second TGF-beta superfamily member.

4. The chimeric protein of claim 1, wherein said chimeric protein is an agonist or antagonist of a third TGF-beta superfamily member.

5. The chimeric protein of claim 1, wherein said core domain comprises a consensus sequence for post-translational modification.

6. The chimeric protein of claim 5, wherein said post-translational modification is glycosylation.

7. The chimeric protein of claim 5, wherein said post-translational modification results in a phosphorylated amino acid, PEGylated amino acid, a farnesylated amino acid, an acetylated amino acid, a biotinylated amino acid, an amino acid conjugated to a lipid moiety, or an amino acid conjugated to an organic derivatizing agent.

8. The chimeric protein of claim 5, wherein said post-translational modification improves stability, solubility, bioavailability, or biodistribution of said chimeric protein.

9. The chimeric protein of claim 1, wherein said core domain comprises a sequence from a Nodal polypeptide.

10. The chimeric protein of claim 8, wherein said core domain comprises a sequence from a murine Nodal polypeptide.

11-15. (canceled)

16. A chimeric TGF-beta superfamily protein comprising a core domain from a first TGF-beta superfamily member and a variable domain from a second TGF-beta superfamily member, wherein said core domain is modified at one or more amino acid positions to generate a consensus sequence for post-translational modification, which post-translationally modified form of said chimeric protein binds to at least one of TGF-beta type 1 receptor and TGF-beta type 2 receptor.

17. The chimeric protein of claim 16, wherein said chimer protein forms a homodimer.

18. The chimeric protein of claim 16, wherein said chimeric protein is an agonist or antagonist of the second TGF-beta superfamily member.

19. The chimeric protein of claim 16, wherein said chimeric protein is an agonist or antagonist of a third TGF-beta superfamily member.

20. The chimeric protein of claim 16, wherein said post-translational modification is glycosylation.

21. The chimeric protein of claim 16, wherein said post-translational modification results in a phosphorylated amino acid, PEGylated amino acid, a farnesylated amino acid, an acetylated amino acid, a biotinylated amino acid, an amino acid conjugated to a lipid moiety, or an amino acid conjugated to an organic derivatizing agent.

22. The chimeric protein of claim 16, wherein said post-translational modification improves stability, solubility, bioavailability, or biodistribution of said chimeric protein.

23. The chimeric protein of claim 16, wherein said core domain comprises a sequence from a Nodal polypeptide.

24. The chimeric protein of claim 23, wherein said core domain comprises a sequence from a murine Nodal polypeptide.

25-28. (canceled)

29. A nucleic acid encoding (i) a chimeric protein that comprises a core domain from a first TGF-beta superfamily member and a variable domain from a second TGF-beta superfamily member, wherein said chimeric protein binds to at least one of TGF-beta type 1 receptor and TGF-beta type 2 receptor, or (ii) a chimeric TGF-beta protein that comprises a core domain from a first TGF-beta superfamily member and a variable domain from a second TGF-beta superfamily member, wherein said core domain is modified at one or more amino acid positions to generate a consensus sequence for post-translational modification, which post-translationally modified form of said chimeric protein binds to at least one of TGF-beta type 1 receptor and TGF-beta type 2 receptor.

30-32. (canceled)

33. A cell transformed with a recombinant polynucleotide comprising a promoter sequence operably linked to a polynucleotide of claim 29.

34. A method of making a chimeric TGF-beta superfamily protein comprising:
a) culturing a cell under conditions suitable for expression of the chimeric TGF-beta superfamily protein, wherein said cell is transformed with a recombinant polynucleotide
comprising a promoter sequence operably linked to a polynucleotide of claim 29; and
b) recovering the chimeric TGF-beta superfamily protein so expressed.

35-40. (canceled)

41. A method for treating a subject having a disorder associated with (i) insufficient bone mineral density, bone loss, bone damage or insufficient bone growth, (ii) abnormal amount, development or metabolic activity of muscle tissue, (iii) neurodegeneration, or (iv) abnormal cell growth and differentiation, comprising administering to the subject an effective amount of a chimeric TGF-beta superfamily protein.

42-55. (canceled)

56. A method for increasing growth of a tissue or decreasing loss of a tissue in a subject, comprising administering to the subject an amount of a chimeric TGF-beta superfamily protein sufficient to increase growth of the tissue or decrease loss of the tissue, wherein the tissue is selected from the group consisting of: bone, cartilage, muscle, and neuron.

57-66. (canceled)

67. A fusion protein comprising the TGF-beta superfamily protein of claim 1, wherein said fusion protein further comprises an Fc of an immunoglobulin or serum albumin.

68. A fusion protein comprising the TGF-beta superfamily protein of claim 16, wherein said fusion protein further comprises an Fc of an immunoglobulin or serum albumin.

69-74. (canceled)

75. A method of promoting regeneration of cardiac tissue damaged by myocardial infarction by administering a therapeutically effective amount of a chimeric protein comprising an amino acid sequence of SEQ ID NO:14 to a subject in need thereof.

76. A modified TGF-beta superfamily protein, wherein a core domain of said protein comprises a modification.

77-89. (canceled)
